(19) United States
(12) Patent Application Publication
(10) Pub. No.: US 2024/0279283 A1
(43) Pub. Date: Aug. 22, 2024

(54) CYCLIC PEPTIDE ANTIBIOTICS

(71) Applicant: Genentech, Inc., South San Francisco, CA (US)

(72) Inventors: Christian Nathaniel CUNNINGHAM, Oakland, CA (US); Jian Mehr-Dean PAYANDEH, Belmont, CA (US); Steven Thomas RUTHERFORD, El Granada, CA (US); Kelly Marie Colvin STOREK, Belmont, CA (US); Patrick C. REID, Kanagawa (JP); Hayato YANAGIDA, Kanagawa (JP); Junichi NISHIKAWA, Kanagawa (JP)

(73) Assignee: Genentech, Inc., South San Francisco, CA (US)

(21) Appl. No.: 18/566,257

(22) PCT Filed: Jun. 1, 2022
(86) PCT No.: PCT/US2022/031732

§ 371 Date: Dec. 1, 2023

Related U.S. Application Data
(60) Provisional application No. 63/196,374, filed on Jun. 3, 2021.

(51) Int. Cl. C07K 7/64; A61K 38/12; A61K 45/06

(52) U.S. Cl. CPC C07K 7/64; A61K 38/12; A61K 45/06

(57) ABSTRACT

Provided herein are antibacterial compound peptides active against Gram-negative bacteria. In various embodiments, the compounds act by inhibition of BAM A membrane protein. Pharmaceutical compositions and methods for treatment using the compounds described herein are also provided.

### CROSS REFERENCE TO RELATED APPLICATIONS

This application claims the benefit of and priority to U.S. Provisional Patent Application No. 63/196,374, filed 3 Jun. 2021, the content of which is incorporated herein by reference in its entirety.

### BACKGROUND

Most Gram-negative bacteria are inherently more resistant to antibacterials than Gram-positive bacteria and currently pose a problem in anti-infective therapy. Initially, this ‘intrinsic resistance’ was attributed to the outer membrane (OM) permeation barrier. However, OM permeability alone is not a sufficient explanation because most drug molecules can even equilibrate, across the rather impermeable OM of P. aeruginosa, in less than a minute. (H. Nikado, Antimicrob. Agent Chemother. 1989 33(11):1831) The intrinsic drug resistance of Gram-negative bacteria has been shown to result from the cooperation between the OM barrier and the expression of broad-specificity multidrug efflux pumps (H. I. Zgurskaya and H. Nikado, Proc. Nat. Acad. Sci. 1999 96(13):7190). Gram-negative bacteria also possess drug-specific efflux pumps that mediate resistance to certain classes of antibacterials. (X.-Z. Li and H. Nikaido, Drugs 2004 64:159-204).

### SUMMARY OF THE DISCLOSURE

Described herein are novel cyclic peptides useful for treating microbial infections. In various embodiments, the present disclosure provides macrocyclic peptides for the treatment of bacterial infections. In various embodiments, the present disclosure provides classes and subclasses of chemical compounds for the treatment of Gram-negative bacterial infections. The macrocyclic compounds act by inhibiting folding and insertion of the nascent proteins into the OM that is mediated by the OPM BAM complex.

In some embodiments of the invention there is provided a cyclic dodecapeptide of Formula (I), or a pharmaceutically acceptable salt, solvate, or stereoisomer thereof:

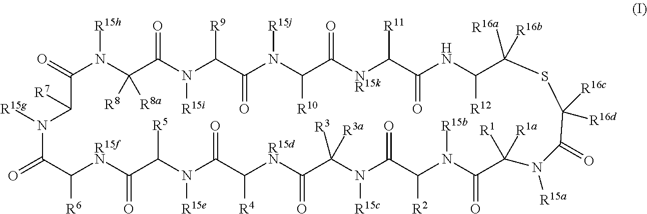

wherein:
   R¹ is C₁₋₆alkyl, hydroxyl-C₁₋₆alkyl or optionally substituted benzyl;
   R^{1a} is hydrogen or C₁₋₆alkyl;
   R² is hydrogen, C₁₋₆alkyl, or optionally substituted (imidazol-4-yl)methyl;
   R³ is bicyclic aryl, bicyclic heteroaryl, C₁₋₆alkyl, or hydroxyl-C₁₋₆alkyl wherein the bicyclic aryl and bicyclic heteroaryl is optionally substituted once or twice with C₁₋₆alkyl, hydroxyl or halo;
   R^{3a} is hydrogen or C₁₋₆alkyl;
   R⁴ is C₁₋₆ alkyl or optionally substituted (imidazol-4-yl)methyl;
   R⁵ is hydrogen, C₁₋₆alkyl, hydroxyl-C₁₋₆alkyl, optionally substituted (imidazol-4-yl)methyl or optionally substituted guanidino-C₂₋₄alkyl;
   R⁶ is hydroxyl-C₁₋₆alkyl or amino-C₁₋₆alkyl;
   R⁷ is C₁₋₆alkyl, hydroxyl-C₁₋₆alkyl or optionally substituted guanidino-C₂₋₄alkyl;
   R⁸ is optionally substituted benzyl;
   R^{8a} is hydrogen or C₁₋₆alkyl;
   R⁹ is optionally substituted benzyl or optionally substituted 1H-imidazol-5-ylmethyl;
   R¹⁰ is C₁₋₆alkyl, amino-C₁₋₆alkyl, optionally substituted 1H-imidazol-5-ylmethyl, optionally substituted guanidino-C₂₋₄alkyl or optionally substituted C₁₋₂alkyl-carboxamide;
   R¹¹ is optionally substituted 1-C₁₋₆alkyl-1H-imidazol-5-yl)methyl benzyl or wherein R^{b} is hydrogen, or C₁₋₆alkyl;
   R¹² is optionally substituted N-(3-amino-3-oxo-(CH₂)₂₋₃)carboxamide;
   R^{15a}, R^{15b}, R^{15c}, R^{15d}, R^{15f}, R^{15g}, R^{15h}, R¹⁵ⁱ, R^{15j}, R^{15k}, R^{16a}, R^{16b}, R^{16c} and R^{16d} each independently in each occurrence hydrogen, or C₁₋₆alkyl.

In other embodiments of the invention there is provided a cyclic tetradecapeptide of Formula (II), or a pharmaceutically acceptable salt, solvate, or stereoisomer thereof,

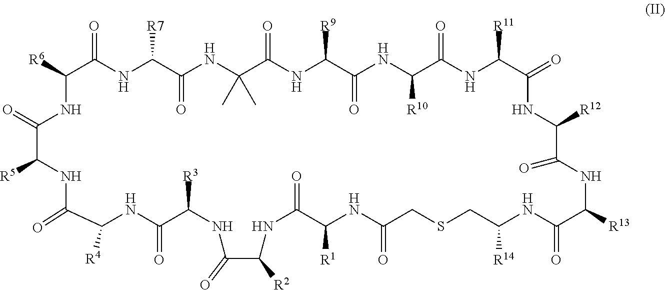

wherein:
   R¹ is C₁₋₆alkyl, hydroxyl-C₁₋₆alkyl or optionally substituted benzyl;
   R^{1a} is hydrogen or C₁₋₆alkyl;
   R² is hydrogen, C₁₋₆alkyl or optionally substituted (imidazol-4-yl)methyl;
   R³ is bicyclic aryl, bicyclic heteroaryl, C₁₋₆alkyl or hydroxyl-C₁₋₆alkyl wherein the bicyclic aryl and bicyclic heteroaryl are optionally substituted once or twice with C₁₋₆alkyl, hydroxyl or halo;
   R^{3a} is hydrogen or C₁₋₆alkyl;
   R⁴ is C₁₋₆alkyl or optionally substituted (imidazol-4-yl)methyl;
   R⁵ is hydrogen, C₁₋₆alkyl, hydroxyl-C₁₋₆alkyl, optionally substituted (imidazol-4-yl)methyl or optionally substituted guanidino-C₂₋₄alkyl;
   R⁶ is hydroxyl-C₁₋₆alkyl or amino-C₁₋₆alkyl;
   R⁷ is C₁₋₆alkyl, hydroxyl-C₁₋₆alkyl or optionally substituted guanidino-C₂₋₄alkyl;
   R⁸ is optionally substituted benzyl;
   R^{8a} is hydrogen or C₁₋₆alkyl;
   R⁹ is optionally substituted benzyl or optionally substituted (imidazol-4-yl)methyl;
   R¹⁰ is C₁₋₆alkyl, amino-C₁₋₆alkyl, optionally substituted (imidazol-4-yl)methyl, optionally substituted guanidino-C₂₋₄alkyl, or optionally substituted C₁₋₂alkyl-carboxamide;
   R¹¹ is optionally substituted benzyl;
   R¹² is 1H-indolyl-3-methyl or optionally substituted benzyl;
   R¹³ is optionally substituted benzyl;
   R¹⁴ is optionally substituted N-(3-amino-3-oxo-(CH₂)₂₋₃)carboxamide;
   R^{b}, R^{c}, R^{d}, R^{e}, R^{f}, R^{15a}, R^{15b}, R^{15c}, R^{15d}, R^{15e}, R^{15f}, R^{15g}, R^{15h}, R¹⁵ⁱ, R^{15j}, R^{15k}, R^{15l}, R^{15m}, R¹⁵ⁿ, R^{16a}, R^{16b}, R^{16c} and R^{16d} are in each occurrence independently hydrogen or C₁₋₆alkyl;
   R^{a} is hydrogen, C₁₋₆alkyl, hydroxyl or halo;
   n is 0 to 2;
   q is 0 or 1; and,
   r is 1 or 2.

### BRIEF DESCRIPTION OF THE FIGURES

FIGS. 1a and 1b—Programs used for preparation of Example 1 and Example 2 in an automated peptide synthesizer

FIGS. 2a and 2b—Programs used for preparation of Example 3 and Example 4 in an automated peptide synthesizer

FIGS. 3a and 3b—Programs used for preparation of Example 5 and Example 6 in an automated peptide synthesizer

### DETAILED DESCRIPTION OF THE DISCLOSURE

Antibiotic resistance is a serious and growing phenomenon in contemporary medicine and has emerged as a major public health concern in the 21st century posing a significant obstacle to therapy. Many Gram-negative (GN) bacteria are more inherently resistant to antibacterials than Gram-positive bacteria. This enhanced resistance is in part due to the cell wall. Gram-positive bacteria have cell walls made of a thick layer of peptidoglycan surrounding a cytoplasmic lipid membrane. In contrast, the cell walls of gram-negative bacteria contain only a thin layer of peptidoglycan, but they also have an outer membrane (OM) that is absent in gram-positive bacteria. The outer membrane of Gram-negative bacteria provides rigidity to the cell and serves as a permeability barrier to cytotoxic molecules including antibiotics. In addition the OM contains integral outer membrane proteins (OMPs), porins, that have a characteristic β-barrel structure and perform essential functions, including—the efflux of toxic molecules from the cell. Thus the presence of BamA in the outer membrane presents a potential target for small molecule inhibitors which inhibit formation of antibiotic efflux pumps. (K. M. Lehman and M. Grabowicz, Antibiotics 2019 8:163)

A library cyclic peptides was prepared using mRNA display. (Roberts, R. W. and Szostak, Proc. Nat. Acad. Sci. USA 1997 94:12297, Sohrabi, C. et al., Nature Rev. Chem. 2020 4:90; Josephson, K et al., Drug Discov. Today, 2014 19(4):388). A thioether-macrocyclic peptide library was constructed by using N-chloroacetyl D-phenylalanine (ClAc-F) as an initiator in a genetically reprogrammed in vitro translation system (Kashiwagi, K and Reid, P., WO2011/049157). The resulting library of macrocyclic peptides was screened resulting in the identification of peptide targeting Bam A 1 and peptide targeting Bam A 2.

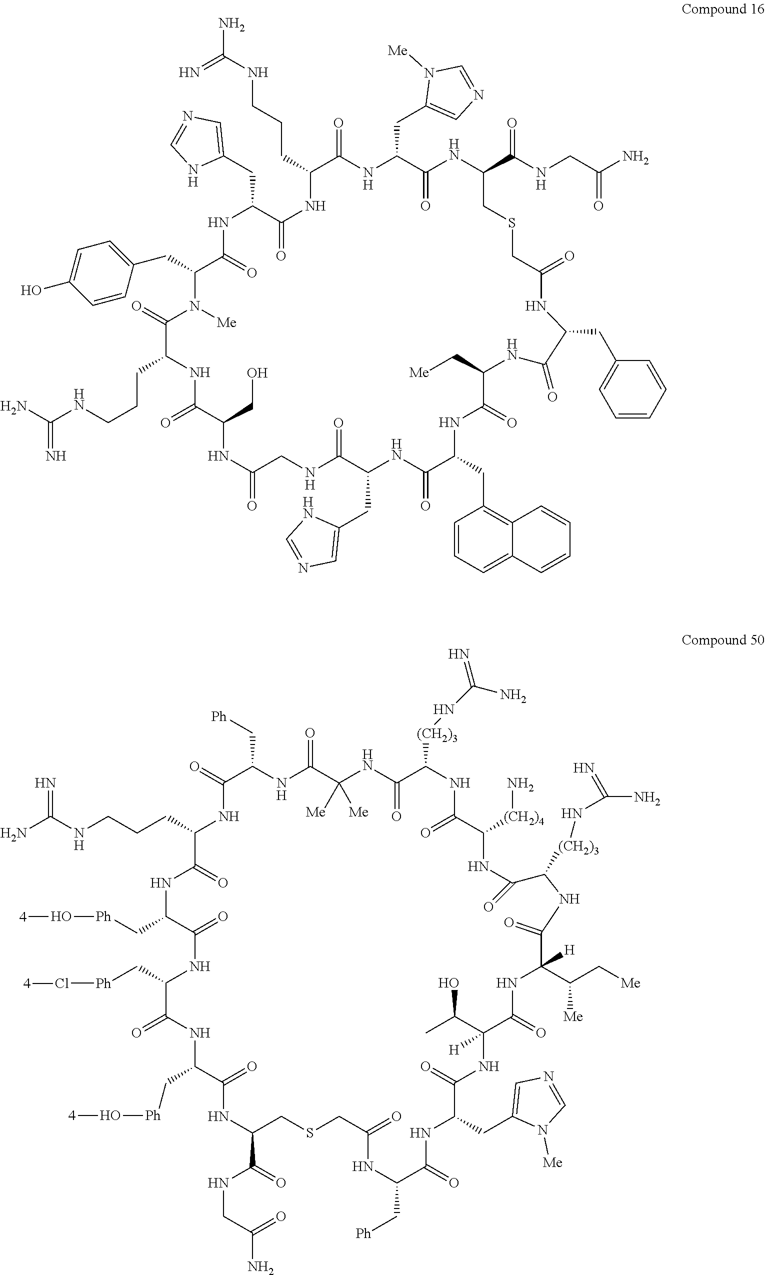

### Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Alkyl” refers to an optionally substituted straight-chain, or optionally substituted branched-chain saturated hydrocarbon monoradical having from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkylene” refers to a straight or branched divalent hydrocarbon chain. Unless stated otherwise specifically in the specification, an alkylene group may be optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen.

“Alkoxy” refers to a radical of the formula —ORₐ where Rₐ is an alkyl radical as defined herein. Unless stated otherwise specifically in the specification, an alkoxy group may be optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted as described below, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen or methyl. In some embodiments, the aryl is optionally substituted with halogen.

“Halo” or “halogen” refers to bromo, chloro, fluoro, or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Aminoalkyl” refers to an alkyl radical, as defined above, that is substituted by one or more —NH₂, e.g., —CH₂NH₂, —CH₂CH₂NH₂, —CH₂CH₂CH₂NH₂, —CH(NH₂)CH₃, —CH₂CH(NH₂)CH₃, —CH(NH₂)CH₂CH₃, and the like.

“Hydroxyalkyl” refers to an alkyl radical, as defined above, that is substituted by one or more —OH, e.g., —CH₂OH, —CH₂CH₂OH, —CH₂CH₂CH₂OH, —CH(OH)CH₃, —CH₂CH(OH)CH₃, —CH(OH)CH₂CH₃, and the like.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted as described below, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen or methyl. In some embodiments, the heteroaryl is optionally substituted with halogen.

The following nomenclature is used herein.

Optionally substituted benzyl refers to benzyl optionally substituted with C₁₋₆ alkyl, hydroxyl, C₁₋₃ alkoxy, halo. C₁₋₆ haloalkyl or C₁₋₆ haloalkyloxy.

1H-imidazol-5-ylmethyl refers to (ii) wherein R^{b} is hydrogen and optionally substituted (imidazol-4-yl)methyl refers to (ii) wherein R^{b} is hydrogen or C₁₋₆ alkyl. Both (3-methylimidazol-4-yl)methyl and 1-methyl-1H-imidazol-5-yl)methyl refer to (iib) wherein R^{b} is methyl.

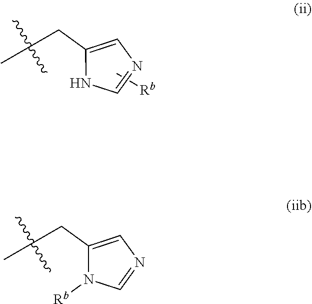

Guanidino-C₂₋₄alkyl refers to a moiety —(CH₂)₂₋₄—NH(C═NH)NH₂; optionally substituted guanidino-C₂₋₄alkyl refers to a moiety —(CH₂)ₚ—NR^{c}(C═NR^{c})N(R^{c})₂ (iii) wherein R^{c} is independently in each occurrence hydrogen or C₁₋₆alkyl and p is 0 to 2, preferably R^{c} is hydrogen or methyl and p is 1.

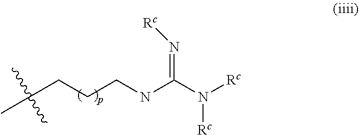

Optionally substituted C₁₋₂alkyl-carboxamide (iv) refers to 3-amino-3-oxo-propyl (—(CH₂)₂C(═O)N(R^{d})₂) and 2-amino-2-oxo-ethyl refer to (—CH₂C(═O)N(R^{d})₂) respectively wherein R^{d} is independently in each occurrence hydrogen or C₁₋₆alkyl and preferably R^{d} is hydrogen or methyl and q is 0 or 1.

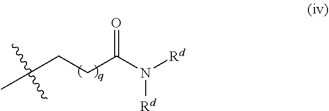

N-(3-amino-3-oxo-(CH₂)₂₋₃)carboxamide-refers to (v) wherein r is 1 or 2 and R^{e} hydrogen. N-(3-amino-3-oxo-propyl)carboxamide refers —C(═O)NR^{e}(CH₂)₂C(═O)N(R^{e})₂ and N-(2-amino-2-oxo-ethyl)carboxamide refers to —C(═O)NR^{e}CH₂C(═O)N(R^{e})₂ wherein R^{e} is hydrogen. When these moieties are optionally substituted R is independently C₁₋₆ alkyl or hydrogen

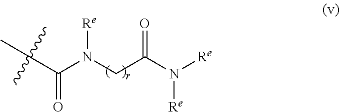

1H-indolyl-3-methyl refers to (vi) wherein R^{f} is hydrogen and optionally substituted 1H-indolyl-3-methyl refers to (vi) wherein R^{f} is hydrogen or C₁₋₆alkyl;

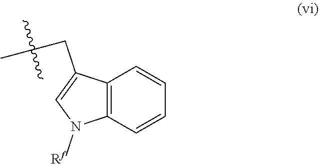

The term aromatic amino acid refers to a compound of formula H₂NCH(CH₂R^{a}) CO₂H wherein R^{a} is benzyl, phenyl, 2-phenethyl, pyridinyl, pyrimidinyl, pyrazinyl or pyridazinyl wherein the aromatic ring is optionally substituted with 1 to 3 groups independently selected from C₁₋₆ alkyl, halogens, C₁₋₆-haloalkyl, hydroxyl, C₁₋₆-alkoxy, C₁₋₆-haloalkoxy, C₁₋₆-thioalkyl, C₁₋₆-alkylsulfonyl and cyano. An N-methyl-aromatic amino acid is a compound of formula MeHNCH(CH₂R^{a}) CO₂H.

The term aliphatic amino acid refers to a compound of formula H₂NCH(CH₂R^{a}) CO₂H wherein R^{a} is hydrogen or C₁₋₆ straight or branched. alkyl, CiAalkoxy-C₁₋₂alkyl or methionine.

The term basic amino acid refers to a compound of formula H₂NCH(CH₂R^{a}) CO₂H wherein R^{a} is C₁₋₆-aminoalkyl, guanidino-C₂₋₄alkyl or optionally substituted C₂₋₄alkyl-(guanidine) wherein R^{c} is independent hydrogen or C₁₋₃alkyl, pyrrolidin-3-yl.

The term heteroaryl amino acid refers to histidine, tryptophan, N-alkyl-histidine or N-alkyl tryptophan wherein the alkyl group is 1-3 carbons.

The term polar amino acid refers to serine, threonine, cysteine, asparagine, or glutamine.

Abbreviations used herein include 4-ClPh (4-chlorophenyl); F4C (4-Chloro-L-phenylalanine); 4-HO-Ph (4-hydroxyphenyl); Boc (t-butoxycarbonyl; Pbf (2,2,4,6,7-pentamethyldihydrobenzofuran-5-sulfonyl); Trt (Trityl); Abu ((S)-2-Aminobutyric acid); H3Me (3-Methyl-L-histidine); MeY (N-Methyl-L-tyrosine); MeF (N-Methyl-L-phenylalanine); TFA (trifluoroacetic acid); MeCN (acetonitrile); TEA (triethylamine); DCM (dichloromethane); DMF (N,N-dimethylformamide) The terms “treat,” “prevent,” “ameliorate,” and “inhibit,” as well as words stemming therefrom, as used herein, do not necessarily imply 100% or complete treatment, prevention, amelioration, or inhibition.

Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. Also, for purposes herein, “treatment,” “prevention,” “amelioration,” or “inhibition” encompass delaying the onset of the disorder, or a symptom or condition thereof.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

The term as defined hereinabove refers the broadest definition described in the summary of the invention or to the larger embodiment to which it refers “Prodrug” as the term is used herein means a compound with one or more moieties that can be metabolized in vivo to produce the active drug. For example, some prodrugs are metabolized in vivo by esterases or by other mechanisms to active drugs. Examples of prodrugs and their uses are well known in the art (see, e.g., Berge et al. (1977) “Pharmaceutical Salts”, J. Pharm. Sci. 66:1-19). Prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran, or methanol. In addition, the compounds provided herein can exist in unsolvated as well as solvated forms. In general, the solvated forms are considered equivalent to the unsolvated forms for the purposes of the compounds and methods provided herein.

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

In another aspect compounds are hydrates or metabolites of any of the aforementioned compounds. In another aspect are pharmaceutical compositions comprising any of the aforementioned compounds together with a pharmaceutically acceptable excipient.

### Compounds

In one embodiment of the invention there is provided a cyclic dodecapeptide of Formula (I), or a pharmaceutically acceptable salt, solvate, or stereoisomer thereof as disclosed above in the Summary of the Disclosure.

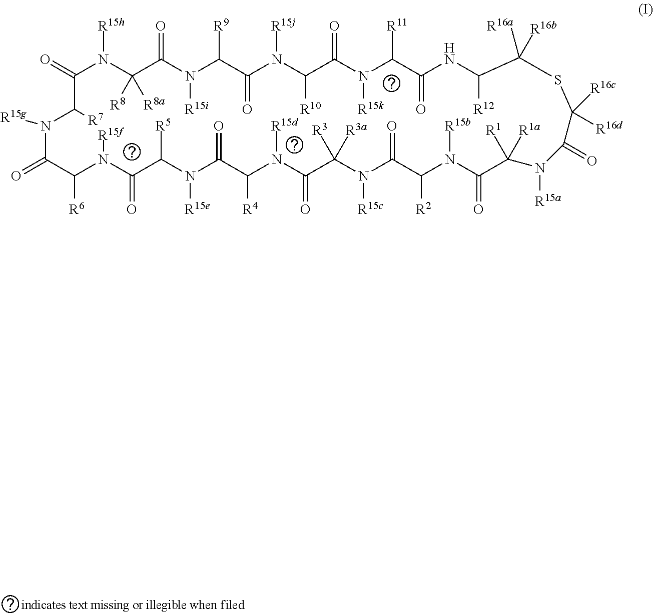

In one embodiment of a compound of Formula (I), R¹² is N-(2-amino-2-oxo-ethyl)carboxamide, R^{1a}, R^{3a} and R^{8a} are hydrogen, R⁴ and R⁹ are 1H-imidazol-5-ylmethyl and R² is C₁₋₆ alkyl. In one embodiment of a compound of Formula (I), R² is ethyl, isopropyl or sec-butyl.

In some embodiments of a compound of Formula (I), R¹ is optionally substituted benzyl. In some embodiments of a compound of Formula (I), R¹ is unsubstituted benzyl, hydroxybenzyl or chlorobenzyl. In some embodiments of a compound of Formula (I), R¹ is 4-hydroxybenzyl or 4-chlorobenzyl. In some embodiments of a compound of Formula (I), R¹ is C₁₋₆ alkyl. In some embodiments of a compound of Formula (I), R¹ is butyl. In some embodiments of a compound of Formula (I), R¹ is C₁₋₆ hydroxyalkyl or hydroxymethyl. In some embodiments of a compound of Formula (I), R¹ is hydroxymethyl.

In some embodiments of a compound of formula (I) R³ is bicyclic aryl, bicyclic heteroaryl, C₁₋₆alkyl, or hydroxyl-C₁₋₆alkyl wherein the bicyclic aryl and bicyclic heteroaryl is optionally substituted once or twice with C₁₋₆alkyl, hydroxyl or halo. In some embodiments R³ is 1-naphthylmethyl either substituted by one to three halogens; or unsubstituted 1-naphthylmethyl. In some embodiments R³ is 2-naphthmethyl either substituted by one to three halogens; or unsubstituted 2-naphthmethyl. In some embodiments R³ is optionally substituted-3-indolylmethyl wherein R^{f} is hydrogen or C₁₋₆alkyl. In some embodiments R³ is 3-indolylmethyl. In some embodiments R³ is N-substituted-3-indolylmethyl wherein R^{f} is methyl. In some embodiment R³ is optionally substituted benzyl. In some embodiment R³ is 4-phenylphenyl)methyl.

In some embodiments of a compound of formula (I), R⁴ is C₁₋₆alkyl or optionally substituted (imidazol-4-yl)methyl. In some embodiments of a compound of formula (I), R⁴ is 4-(imidazolyl)methyl optionally substituted by C₁₋₆alkyl. In some embodiments of a compound of formula (I), R⁴ is 4-(imidazolyl)methyl. In some embodiments of a compound of formula (I) R⁴ is 4-(imidazolyl)methyl with a N-substituted methyl. In some embodiments of a compound of formula (I) R⁴ is C₁₋₆alkyl. In some embodiments of a compound of formula (I) R⁴ is methyl.

In some embodiments of a compound of formula (I), R⁵ is hydrogen. In some embodiments of a compound of formula (I), R⁵ is hydroxy-C₁₋₆alkyl. In some embodiments of a compound of formula (I), R⁵ is hydroxymethyl. In some embodiments of a compound of formula (I), R⁵ is C₁₋₆alkyl. In some embodiments of a compound of formula (I), R⁵ is methyl. In some embodiments of a compound of formula (I) R⁵ is optionally substituted 3-guanido-C₂₋₄alkyl wherein R^{c} is independently is hydrogen, or C₁₋₆alkyl and p is 2-4. In some embodiments of a compound of formula (I), R⁵ is 3-guanidopropyl.

In some embodiments of a compound of formula (I), R⁶ is hydroxyl-C₁₋₆alkyl. In some embodiments of a compound of formula (I), R⁶ is hydroxymethyl or hydroxyethyl. In some embodiments of a compound of formula (I), R⁶ is 1-hydroxy-ethyl. In some embodiments of a compound of formula (I), R⁶ is amino-C₁₋₆ alkyl. In some embodiments of a compound of formula (I), R⁶ is 4-aminobutyl. In some embodiments of a compound of formula (I), R⁶ is optionally substituted guanidino-C₂₋₄alkyl wherein R^{c} is independently is hydrogen or C₁₋₆alkyl or optionally substituted 3-guanido-propyl. In some embodiments of a compound of formula (I), R⁵ is 3-guanidopropyl.

In some embodiments of a compound of formula (I), R⁷ is optionally substituted 3-guanido-C₂₋₄alkyl wherein R^{c} is independently is hydrogen or C₁₋₆alkyl. In some embodiments of a compound of formula (I), R⁵ is 3-guanidopropyl. In some embodiments of a compound of formula (I), R⁷ is C₁₋₆alkyl. In some embodiments of a compound of formula (I), R⁷ is methyl or ethyl. In some embodiments of a compound of formula (I), R⁷ is methyl. In some embodiments of a compound of formula (I), R⁷ is ethyl. In some embodiments of a compound of formula (I), R⁷ is hydroxylC₁₋₆alkyl. In some embodiments of a compound of formula (I), R⁷ is 1-hydroxyethyl.

In some embodiments of a compound of formula (I), R is optionally substituted benzyl. In some embodiments of a compound of formula (I), R⁹ is hydroxybenzyl. In some embodiments of a compound of formula (I), R⁹ is 4-hydroxybenzyl. In some embodiments of a compound of formula (I), R⁹ is halo substituted benzyl. In some embodiments of a compound of formula (I), R⁹ is benzyl substituted by 1 or 2 halogens. In some embodiments of a compound of formula (I), R⁹ is benzyl substituted by 1 or 2 chlorines. In some embodiments of a compound of formula (I), R⁹ is 4-chlorobenzyl.

In some embodiments of a compound of formula (I), R⁹ is optionally substituted (imidazol-4-yl)methyl. In some embodiments of a compound of formula (I), R⁹ is 4-(imidazolyl)methyl optionally substituted by C₁₋₆alkyl. In some embodiments of a compound of formula (I), R⁴ is 4-(imidazolyl)methyl. In some embodiments of a compound of formula (I), R⁴ is 4-(imidazolyl)methyl with a N-substituted methyl. In some embodiments of a compound of formula (I), R⁹ is C₁₋₆alkyl. In some embodiments of a compound of formula (I), R⁹ is methyl.

In some embodiments of a compound of formula (I), R¹⁰ is optionally substituted 3-guanido-C₁₋₆alkyl wherein R^{c} is independently is hydrogen or C₁₋₆alkyl. In some embodiments of a compound of formula (I), R⁵ is 3-guanidopropyl. In some embodiments of a compound of formula (I), R¹⁰ is —CH₂(CH₂)_{q}C(═O)NH₂ wherein q is 0 or 1. In some embodiments of a compound of formula (I), 3-oxo-3-aminopropyl. In some embodiments of a compound of formula (I), R¹⁰ is amino-C₁₋₆alkyl. In some embodiments of a compound of formula (I), R¹⁰ is aminomethyl, 2-aminoethyl or 4-aminobutyl. In some embodiments R¹⁰ is 3-(methylimidazol-4-yl)methyl (iib wherein R^{b} is methyl). In some embodiments of a compound of formula (I), R¹⁰ is C₁₋₆alkyl. In some embodiments of a compound of formula (I), R¹⁰ is ethyl.

In some embodiments of a compound of formula (I), R¹¹ is 4-(imidazolyl)methyl optionally substituted with C₁₋₆alkyl. In some embodiments of a compound of formula (I), R¹¹ is C₁₋₆alkyl. In some embodiments of a compound of formula (I), R¹¹ is 4-(imidazolyl)methyl with a N-substituted methyl. In some embodiments of a compound of formula (I), R¹¹ is 3-(methylimidazol-4-yl)methyl (iib wherein R^{b} is methyl). In some embodiments of a compound of formula (I), R¹¹ is 4-(imidazolyl)methyl.

In some embodiments of a compound of formula (I), R^{b}, R^{c}, R^{d}, R^{e}, R^{f}, R^{1a}, R^{3a}, R^{8a}, R^{15a}, R^{15b}, R^{15c}, R^{15d}, R^{15e}, R^{15f}, R^{15g}, R¹⁵ⁱ, R^{15j}, R^{15k}, R^{16a}, R^{16b}, R^{16c} and R^{16d} are hydrogen or C₁₋₆ alkyl; in some embodiments R^{b}, R^{c}, R^{d}, R^{e}, R^{f}, R^{1a}, R^{3a}, R^{8a}, R^{15a}, R^{15b}, R^{15c}, R^{15d}, R^{15e}, R^{15f}, R^{15g}, R¹⁵ⁱ, R^{15j}, R^{15k}, R^{16a}, R^{16b}, R^{16c} and R^{16d} are hydrogen or methyl. In some embodiments of a compound of formula (I), R^{a} is hydrogen, C₁₋₆alkyl, hydroxyl or halo; in some embodiment R^{a} is chloro. In some embodiments of a compound of formula (I) R^{f} is hydrogen, C₁₋₆alkyl. In some embodiments of a compound of formula (I), R^{f} is hydrogen. In some embodiments of a compound of formula (I), R^{f} is methyl. In some embodiments of a compound of formula (I), n and p are independently from 0 to 2. In some embodiments of a compound of formula (I), n and p are 1 or 0. In some embodiments of a compound of formula (I), n is 1. In some embodiments of a compound of formula (I), n is 0. In some embodiments of a compound of formula (I), q is independently from 0 or 1. In some embodiments of a compound of formula (I), in some embodiments q is 1. In some embodiments of a compound of formula (I), r is independently from 1 or 2. In some embodiments of a compound of formula (I), r is 1.

In some embodiments there is a compound of formula (I) wherein the compound is selected from Table 1.

TABLE 1

| # | Structure | Compound Name | m/e |
|---|---|---|---|
| 1 | 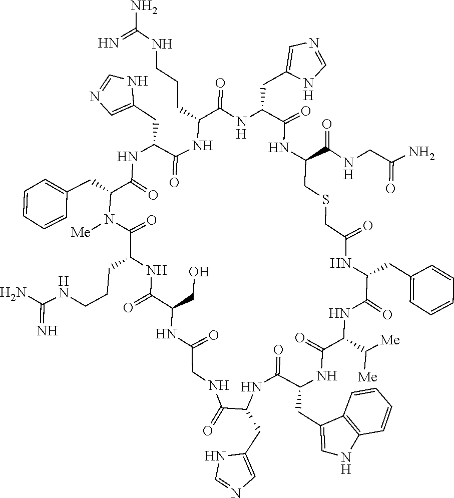 | rac- (3R,6S,9S,12S,15S,18S,21S,27S,30S, 33S,36S)-N-(2-amino-2-oxo-ethyl)- 15,36-dibenzyl-9,18-bis(3- guanidinopropyl)-21- (hydroxymethyl)-6,12,27-tris(1H- imidazol-5-ylmethyl)-30-(1H-indol-3- ylmethyl)-33-isopropyl-16-methyl- 5,8,11,14,17,20,23,26,29,32,35,38- dodecaoxo-1-thia- 4,7,10,13,16,19,22,25,28,31,34,37- dodecazacyclononatriacontane-3- carboxamide | 1678.78 |
| 2 | 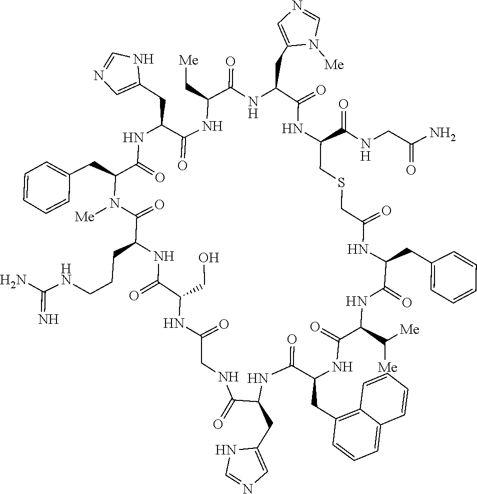 [SMILES: CC[C@@H]1NC(=O)[C@H](Cc2cnc[nH]2)NC(=O)[C@H](Cc2ccccc2)N(C)C(=O)[C@H](CCCNC(=N)N)NC(=O)[C@H](CO)NC(=O)CNC(=O)[C@H](Cc2cnc[nH]2)NC(=O)[C@H](Cc2cccc3ccccc23)NC(=O)[C@H](C(C)C)NC(=O)[C@H](Cc2ccccc2)NC(=O)CSC[C@H](C(=O)NCC(N)=O)NC(=O)[C@H](Cc2cncn2C)NC1=O] | (3R,6S,9S,12S,15S,18S,21S,27S,30S, 33S,36S)-N-(2-amino-2-oxo-ethyl)- 15,36-dibenzyl-9-ethyl-18-(3- guanidinopropyl)-21- (hydroxymethyl)-12,27-bis(1H- imidazol-4-ylmethyl)-33-isopropyl- 16-methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1622.88 |
| 3 | 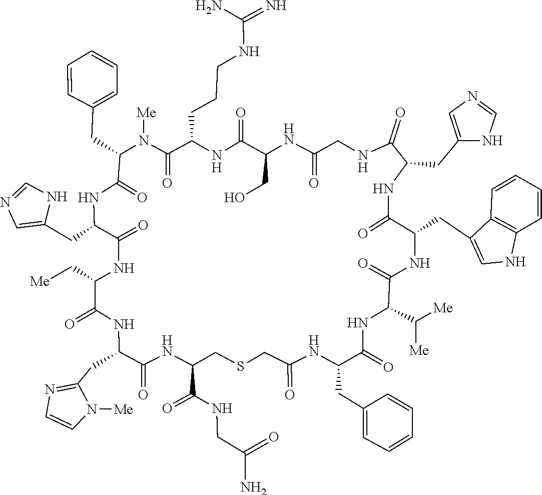 [SMILES: CC[C@@H]1NC(=O)[C@H](Cc2cnc[nH]2)NC(=O)[C@H](Cc2ccccc2)N(C)C(=O)[C@H](CCCNC(=N)N)NC(=O)[C@H](CO)NC(=O)CNC(=O)[C@H](Cc2cnc[nH]2)NC(=O)[C@H](Cc2c[nH]c3ccccc23)NC(=O)[C@H](C(C)C)NC(=O)[C@H](Cc2ccccc2)NC(=O)CSC[C@@H](C(=O)NCC(N)=O)NC(=O)[C@H](Cc2nccn2C)NC1=O] | (3R,6S,9S,12S,15S,18S,21S,27S,30S, 33S,36S)-N-(2-amino-2-oxo-ethyl)- 15,36-dibenzyl-9-ethyl-18-(3- guanidinopropyl)-21- (hydroxymethyl)-12,27-bis(1H- imidazol-4-ylmethyl)-30-(1H-indol-3- ylmethyl)-33-isopropyl-16-methyl-6- [(3-methylimidazol-4-yl)methyl]- 5,8,11,14,17,20,23,26,29,32,35,38- dodecaoxo-1-thia- 4,7,10,13,16,19,22,25,28,31,34,37- dodecazacyclononatriacontane-3- carboxamide | 1621.76 |
| 4 | 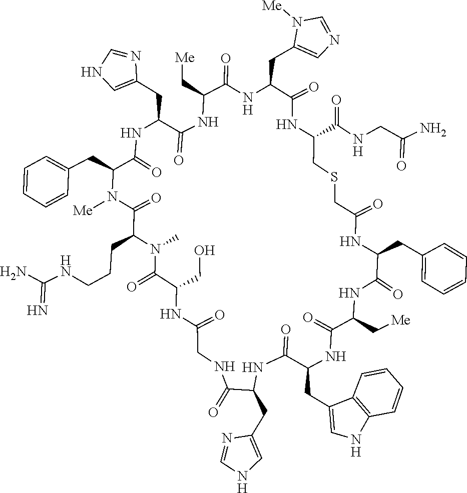 | (3R,6S,9S,12S,15S,18S,21S,27S,30S, 33S,36S)-N-(2-amino-2-oxo-ethyl)- 15,36-dibenzyl-9,33-diethyl-18-(3- guanidinopropyl)-21- (hydroxymethyl)-12,27-bis(1H- imidazol-4-ylmethyl)-30-(1H-indol-3- ylmethyl)-16-methyl-6-[(3- methylimidazol-4-yl)methyl]- 5,8,11,14,17,20,23,26,29,32,35,38- dodecaoxo-1-thia- 4,7,10,13,16,19,22,25,28,31,34,37- dodecazacyclononatriacontane-3- carboxamide | 1607.83 |
| 5 | 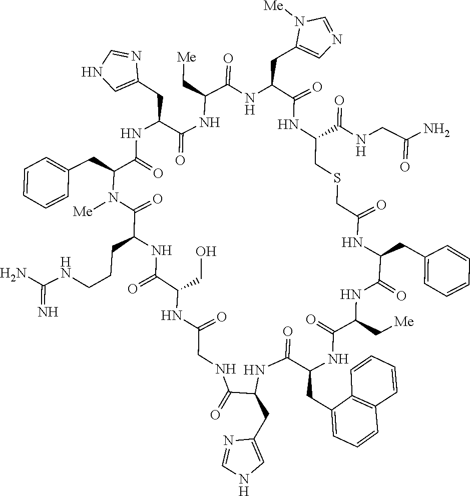 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CC)NC(=O)[C@H](Cc2c[nH]cn2)NC(=O)[C@H](Cc2ccccc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | (3R,6S,9S,12S,15S,18S,21S,27S,30S, 33S,36S)-N-(2-amino-2-oxo-ethyl)- 15,36-dibenzyl-9,33-diethyl-18-(3- guanidinopropyl)-21- (hydroxymethyl)-12,27-bis(1H- imidazol-4-ylmethyl)-16-methyl-6- [(3-methylimidazol-4-yl)methyl]-30- (1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1618.86 |
| 6 | 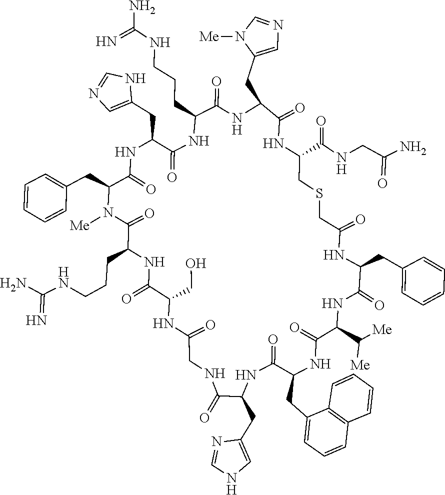 [SMILES: CC(C)[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)NC(=O)[C@H](Cc2cnc[nH]2)NC(=O)[C@H](Cc2ccccc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | (3R,6S,9S,12S,15S,18S,21S,27S,30S, 33S,36S)-N-(2-amino-2-oxo-ethyl)- 15,36-dibenzyl-9,18-bis(3- guanidinopropyl)-21- (hydroxymethyl)-12,27-bis(1H- imidazol-4-ylmethyl)-33-isopropyl- 16-methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1703.8 |
| 7 | 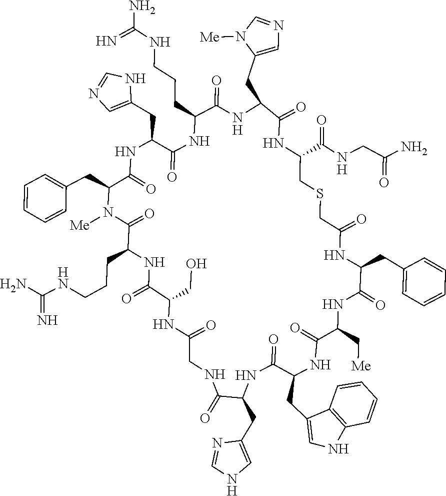 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)NC(=O)[C@H](Cc2cnc[nH]2)NC(=O)[C@H](Cc2ccccc2)N(C)C(=O)[C@H](CCCNC(=N)N)NC(=O)[C@H](CO)NC(=O)CNC(=O)[C@H](Cc2c[nH]cn2)NC(=O)[C@H](Cc2c[nH]c3ccccc23)NC1=O] | (3R,6S,9S,12S,15S,18S,21S,27S,30S, 33S,36S)-N-(2-amino-2-oxo-ethyl)- 15,36-dibenzyl-33-ethyl-9,18-bis(3- guanidinopropyl)-21- (hydroxymethyl)-12,27-bis(1H- imidazol-4-ylmethyl)-30-(1H-indol-3- ylmethyl)-16-methyl-6-[(3- methylimidazol-4-yl)methyl]- 5,8,11,14,17,20,23,26,29,32,35,38- dodecaoxo-1-thia- 4,7,10,13,16,19,22,25,28,31,34,37- dodecazacyclononatriacontane-3- carboxamide | 1678.78 |
| 8 | 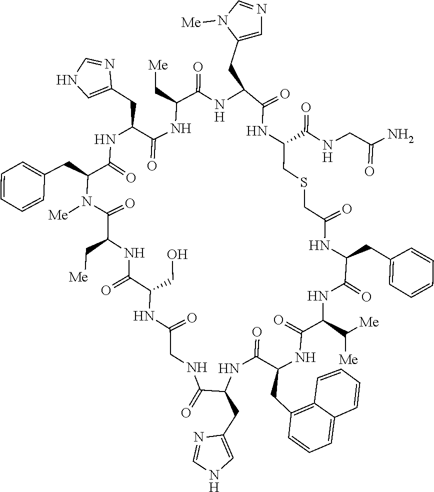 [SMILES: CC[C@@H]1NC(=O)[C@H](Cc2c[nH]cn2)NC(=O)[C@H](Cc2ccccc2)N(C)C(=O)[C@H](CC)NC(=O)[C@H](CO)NC(=O)CNC(=O)[C@H](Cc2c[nH]cn2)NC(=O)[C@H](Cc2cccc3ccccc23)NC(=O)[C@H](C(C)C)NC(=O)[C@H](Cc2ccccc2)NC(=O)CSC[C@@H](C(=O)NCC(N)=O)NC(=O)[C@H](Cc2cncn2C)NC1=O] | (3R,6S,9S,12S,15S,18S,21S,27S,30S, 33S,36S)-N-(2-amino-2-oxo-ethyl)- 15,36-dibenzyl-9,18-diethyl-21- (hydroxymethyl)-12,27-bis(1H- imidazol-4-ylmethyl)-33-isopropyl- 16-methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1561.71 |
| 9 | 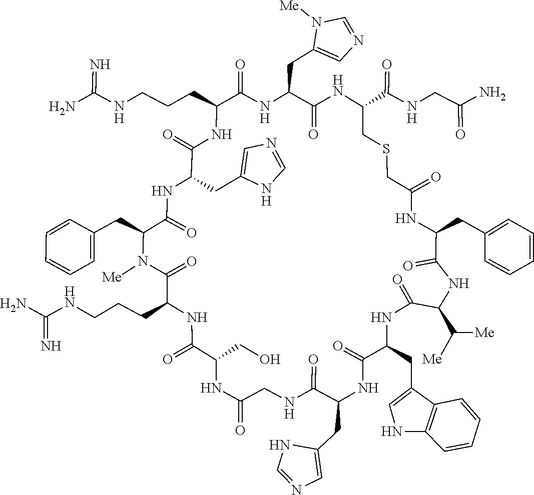 [SMILES: CC(C)[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)NC(=O)[C@H](Cc2cnc[nH]2)NC(=O)[C@H](Cc2ccccc2)N(C)C(=O)[C@H](CCCNC(=N)N)NC(=O)[C@H](CO)NC(=O)CNC(=O)[C@H](Cc2cnc[nH]2)NC(=O)[C@H](Cc2c[nH]c3ccccc23)NC1=O] | rac- (3R,6S,9S,12S,15S,18S,21S,27S,30S, 33S,36S)-N-(2-amino-2-oxo-ethyl)- 15,36-dibenzyl-9,18-bis(3- guanidinopropyl)-21- (hydroxymethyl)-12,27-bis(1H- imidazol-5-ylmethyl)-30-(1H-indol-3- ylmethyl)-33-isopropyl-16-methyl-6- [(3-methylimidazol-4-yl)methyl]- 5,8,11,14,17,20,23,26,29,32,35,38- dodecaoxo-1-thia- 4,7,10,13,16,19,22,25,28,31,34,37- dodecazacyclononatriacontane-3- carboxamide | 1692.80 |
| 10 | 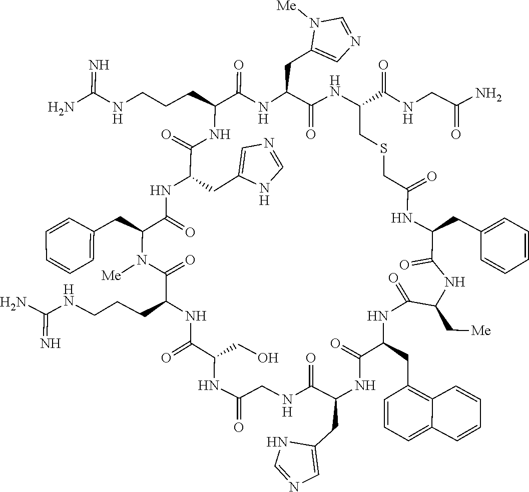 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)NC(=O)[C@H](Cc2cnc[nH]2)NC(=O)[C@H](Cc2ccccc2)N(C)C(=O)[C@H](CCCNC(=N)N)NC(=O)[C@H](CO)NC(=O)CNC(=O)[C@H](Cc2cnc[nH]2)NC(=O)[C@H](Cc2cccc3ccccc23)NC1=O] | rac- (3R,6S,9S,12S,15S,18S,21S,27S,30S,3 3S,36S)-N-(2-amino-2-oxo-ethyl)- 15,36-dibenzyl-33-ethyl-9,18-bis(3- guanidinopropyl)-21- (hydroxymethyl)-12,27-bis(1H- imidazol-5-ylmethyl)-16-methyl-6- [(3-methylimidazol-4-yl)methyl]-30- (1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1689.79 |
| 11 | 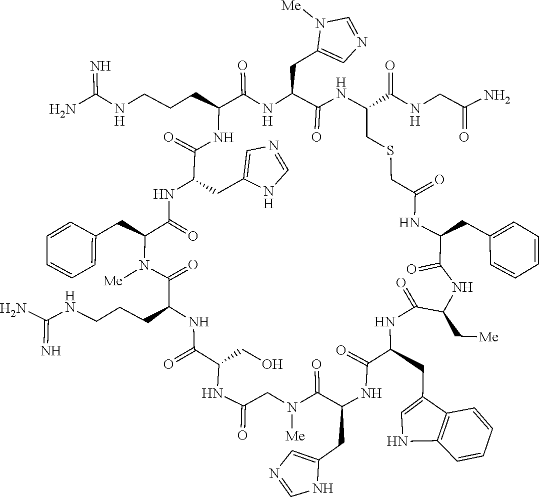 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)NC(=O)[C@H](Cc2cnc[nH]2)NC(=O)[C@H](Cc2ccccc2)N(C)C(=O)[C@H](CCCNC(=N)N)NC(=O)[C@H](CO)NC(=O)CN(C)C(=O)[C@H](Cc2cnc[nH]2)NC(=O)[C@H](Cc2c[nH]c3ccccc23)NC1=O] | rac- (3R,6S,9S,12S,15S,18S,21S,27S,30S, 33S,36S)-N-(2-amino-2-oxo-ethyl)- 15,36-dibenzyl-33-ethyl-9,18-bis(3- guanidinopropyl)-21- (hydroxymethyl)-12,27-bis(1H- imidazol-5-ylmethyl)-30-(1H-indol-3- ylmethyl)-16,25-dimethyl-6-[(3- methylimidazol-4-yl)methyl]- 5,8,11,14,17,20,23,26,29,32,35,38- dodecaoxo-1-thia- 4,7,10,13,16,19,22,25,28,31,34,37- dodecazacyclononatriacontane-3- carboxamide | 1692.80 |
| 12 | 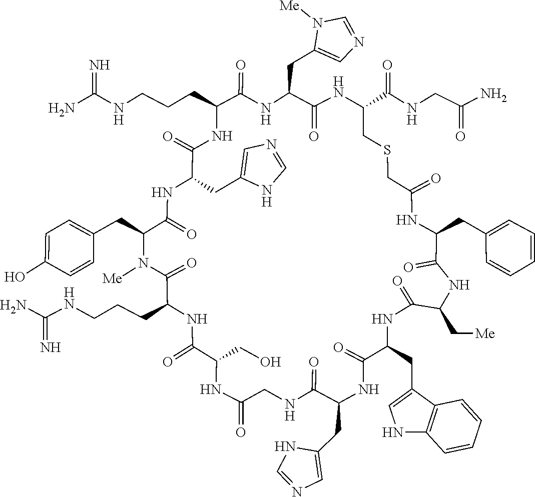 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)NC(=O)[C@H](Cc2cnc[nH]2)NC(=O)[C@H](Cc2ccc(O)cc2)N(C)C(=O)[C@H](CCCNC(=N)N)NC(=O)[C@H](CO)NC(=O)CNC(=O)[C@H](Cc2cnc[nH]2)NC(=O)[C@H](Cc2c[nH]c3ccccc23)NC1=O] | rac- (3R,6S,9S,12S,15S,18S,21S,27S,30S, 33S,36S)-N-(2-amino-2-oxo-ethyl)-36- benzyl-33-ethyl-9,18-bis(3- guanidinopropyl)-21- (hydroxymethyl)-15-[(4- hydroxyphenyl)methyl]-12,27- bis(1H-imidazol-5-ylmethyl)-30-(1H- indol-3-ylmethyl)-16-methyl-6-[(3- methylimidazol-4-yl)methyl]- 5,8,11,14,17,20,23,26,29,32,35,38- dodecaoxo-1-thia- 4,7,10,13,16,19,22,25,28,31,34,37- dodecazacyclononatriacontane-3- carboxamide | 1694.78 |
| 13 | 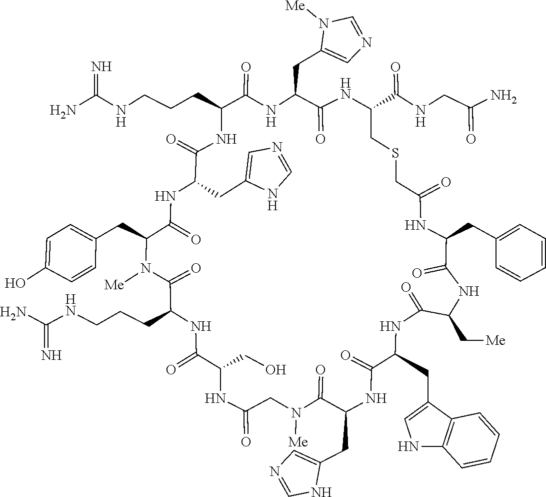 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)NC(=O)[C@H](Cc2cnc[nH]2)NC(=O)[C@H](Cc2ccc(O)cc2)N(C)C(=O)[C@H](CCCNC(=N)N)NC(=O)[C@H](CO)NC(=O)CN(C)C(=O)[C@H](Cc2cnc[nH]2)NC(=O)[C@H](Cc2c[nH]c3ccccc23)NC1=O] | rac- (3R,6S,9S,12S,15S,18S,21S,27S,30S, 33S,36S)-N-(2-amino-2-oxo-ethyl)-36- benzyl-33-ethyl-9,18-bis(3- guanidinopropyl)-21- (hydroxymethyl)-15-[(4- hydroxyphenyl)methyl]-12,27- bis(1H-imidazol-5-ylmethyl)-30-(1H- indol-3-ylmethyl)-16,25-dimethyl-6- [(3-methylimidazol-4-yl)methyl]- 5,8,11,14,17,20,23,26,29,32,35,38- dodecaoxo-1-thia- 4,7,10,13,16,19,22,25,28,31,34,37- dodecazacyclononatriacontane-3- carboxamide | 1708.80 |
| 14 | 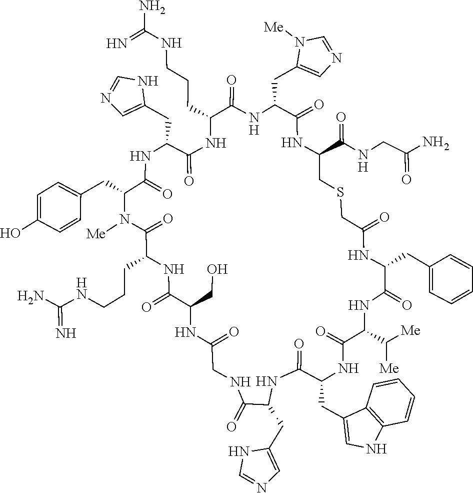 | rac- (3R,6S,9S,12S,15S,18S,21S,27S,30S, 33S,36S)-N-(2-amino-2-oxo-ethyl)-36- benzyl-9,18-bis(3-guanidinopropyl)- 21-(hydroxymethyl)-15-[(4- hydroxyphenyl)methyl]-12,27- bis(1H-imidazol-5-ylmethyl)-30-(1H- indol-3-ylmethyl)-33-isopropyl-16- methyl-6-[(3-methylimidazol-4- yl)methyl]- 5,8,11,14,17,20,23,26,29,32,35,38- dodecaoxo-1-thia- 4,7,10,13,16,19,22,25,28,31,34,37- dodecazacyclononatriacontane-3- carboxamide | 1708.79 |
| 15 | 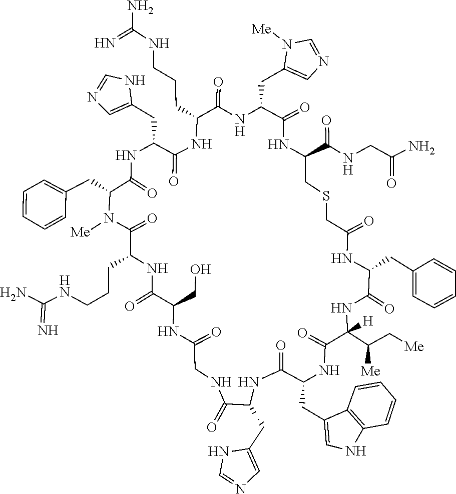 | rac- (3R,6S,9S,12S,15S,18S,21S,27S,30S, 33S,36S)-N-(2-amino-2-oxo-ethyl)- 15,36-dibenzyl-9,18-bis(3- guanidinopropyl)-21- (hydroxymethyl)-12,27-bis(1H- imidazol-5-ylmethyl)-30-(1H-indol-3- ylmethyl)-16-methyl-6-[(3- methylimidazol-4-yl)methyl]- 5,8,11,14,17,20,23,26,29,32,35,38- dodecaoxo-33-[rac-(1S)-1- methylpropyl]-1-thia- 4,7,10,13,16,19,22,25,28,31,34,37- dodecazacyclononatriacontane-3- carboxamide | 1706.82 |
| 16 | 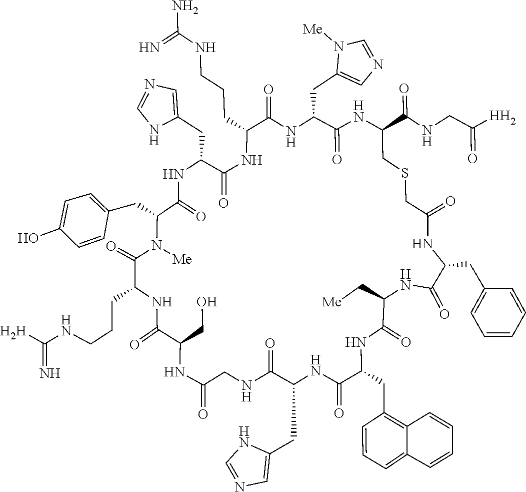 | rac- (3R,6S,9S,12S,15S,18S,21S,27S,30S, 33S,36S)-N-(2-amino-2-oxo-ethyl)-36- benzyl-33-ethyl-9,18-bis(3- guanidinopropyl)-21- (hydroxymethyl)-15-[(4- hydroxyphenyl)methyl]-12,27- bis(1H-imidazol-5-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1705.78 |
| 17 | 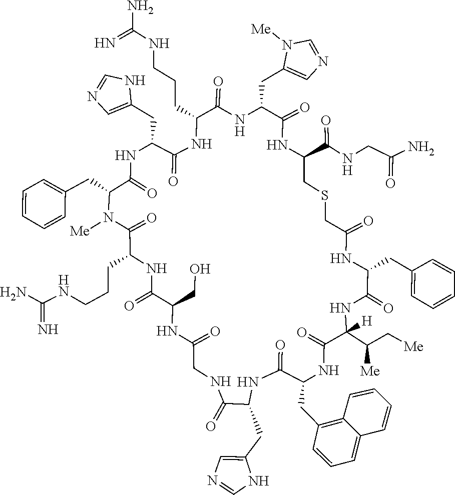 [SMILES: CC[C@@H](C)[C@H]1NC(=O)[C@@H](Cc2ccccc2)NC(=O)CSC[C@H](C(=O)NCC(N)=O)NC(=O)[C@@H](Cc2cncn2C)NC(=O)[C@@H](CCCNC(=N)N)NC(=O)[C@@H](Cc2cnc[nH]2)NC(=O)[C@@H](Cc2ccccc2)N(C)C(=O)[C@@H](CCCNC(=N)N)NC(=O)[C@@H](CO)NC(=O)CNC(=O)[C@@H](Cc2cnc[nH]2)NC(=O)[C@@H](Cc2cccc3ccccc23)NC1=O] | rac- (3R,6S,9S,12S,15S,18S,21S,27S,30S, 33S,36S)-N-(2-amino-2-oxo-ethyl)- 15,36-dibenzyl-9,18-bis(3- guanidinopropyl)-21- (hydroxymethyl)-12,27-bis(1H- imidazol-5-ylmethyl)-16-methyl-6- [(3-methylimidazol-4-yl)methyl]-30- (1-naphthylmethyl)- 5,8,11,14,17,20,23,26,29,32,35,38- dodecaoxo-33-[rac-(1S)-1- methylpropyl]-1-thia- 4,7,10,13,16,19,22,25,28,31,34,37- dodecazacyclononatriacontane-3- carboxamide | 1717.82 |
| 18 | 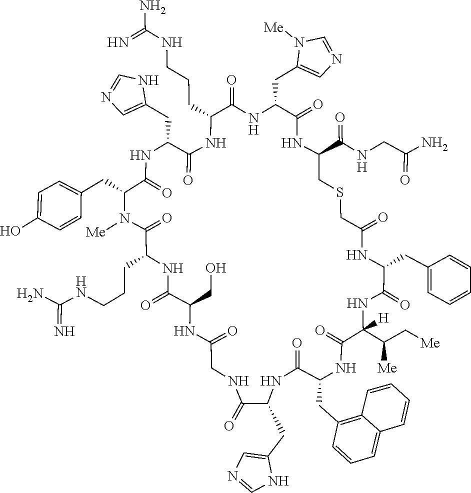 [SMILES: CC[C@@H](C)[C@H]1NC(=O)[C@@H](Cc2ccccc2)NC(=O)CSC[C@H](C(=O)NCC(N)=O)NC(=O)[C@@H](Cc2cncn2C)NC(=O)[C@@H](CCCNC(=N)N)NC(=O)[C@@H](Cc2cnc[nH]2)NC(=O)[C@@H](Cc2ccc(O)cc2)N(C)C(=O)[C@@H](CCCNC(=N)N)NC(=O)[C@@H](CO)NC(=O)CNC(=O)[C@@H](Cc2cnc[nH]2)NC(=O)[C@@H](Cc2cccc3ccccc23)NC1=O] | (3R,6S,9S,12S,15S,18S,21S,27S,30S, 33S,36S)-N-(2-amino-2-oxo-ethyl)-36- benzyl-9,18-bis(3-guanidinopropyl)- 21-(hydroxymethyl)-15-[(4- hydroxyphenyl)methyl]-12,27- bis(1H-imidazol-5-ylmethyl)-16- methyl-6-[(3-methylimidazol-4- yl)methyl]-30-(1-naphthylmethyl)- 5,8,11,14,17,20,23,26,29,32,35,38- dodecaoxo-33-[rac-(1S)-1- methylpropyl]-1-thia- 4,7,10,13,16,19,22,25,28,31,34,37- dodecazacyclononatriacontane-3- carboxamide | 1733.82 |
| 19 | 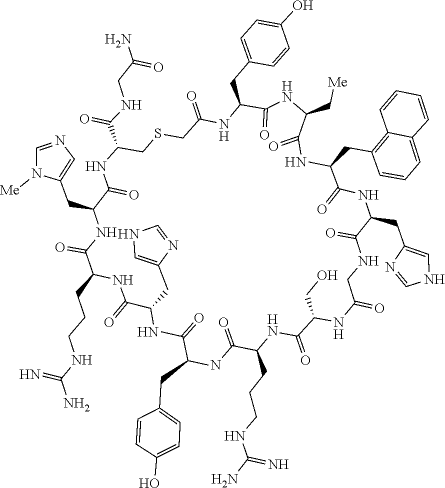 | (3R,6S,9S,12S,15S,18S,21S,27S,30S, 33S,36S)-N-(2-amino-2-oxo-ethyl)-33- ethyl-9,18-bis(3-guanidinopropyl)- 21-(hydroxymethyl)-15,36-bis[(4- hydroxyphenyl)methyl]-12,27- bis(1H-imidazol-4-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1721.78 |
| 20 | 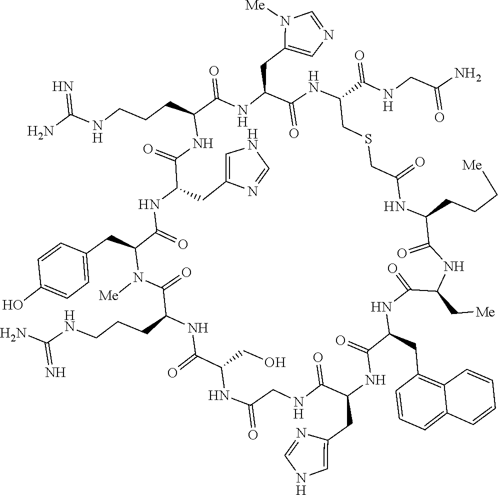 [SMILES: CCCC[C@@H]1NC(=O)CSC[C@@H](C(=O)NCC(N)=O)NC(=O)[C@H](Cc2cncn2C)NC(=O)[C@H](CCCNC(=N)N)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC(=O)[C@H](CC)NC1=O] | (3R,6S,9S,12S,15S,18S,21S,27S,30S, 33S,36S)-N-(2-amino-2-oxo-ethyl)-36- butyl-33-ethyl-9,18-bis(3- guanidinopropyl)-21- (hydroxymethyl)-15-[(4- hydroxyphenyl)methyl]-12,27- bis(1H-imidazol-4-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1671.80 |
| 21 | 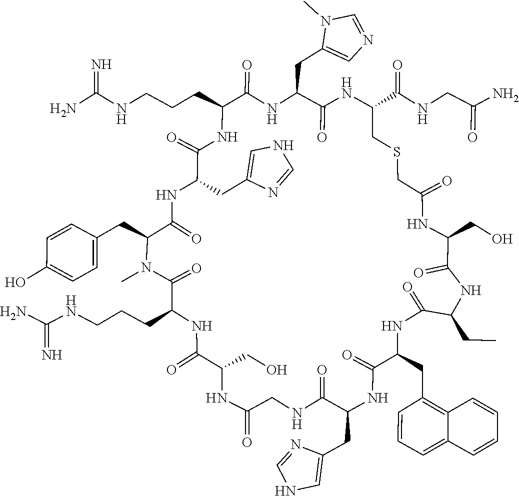 [SMILES: CC[C@@H]1NC(=O)[C@H](CO)NC(=O)CSC[C@@H](C(=O)NCC(N)=O)NC(=O)[C@H](Cc2cncn2C)NC(=O)[C@H](CCCNC(=N)N)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | (3R,6S,9S,12S,15S,18S,21S,27S,30S, 33S,36S)-N-(2-amino-2-oxo-ethyl)-33- ethyl-9,18-bis(3-guanidinopropyl)- 21,36-bis(hydroxymethyl)-15-[(4- hydroxyphenyl)methyl]-12,27- bis(1H-imidazol-4-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1645.75 |
| 22 | 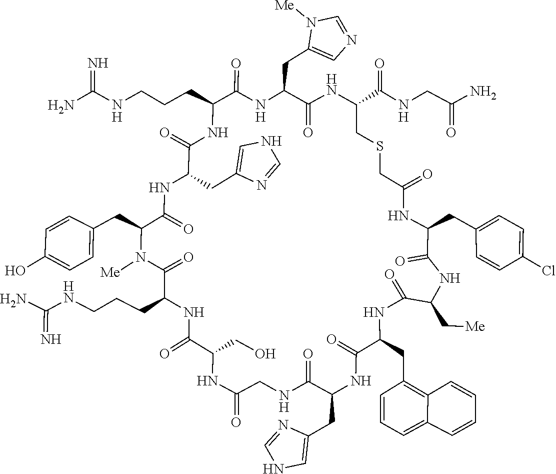 [SMILES: CC[C@@H]1NC(=O)[C@H](Cc2ccc(Cl)cc2)NC(=O)CSC[C@@H](C(=O)NCC(N)=O)NC(=O)[C@H](Cc2cncn2C)NC(=O)[C@H](CCCNC(=N)N)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | (3R,6S,9S,12S,15S,18S,21S,27S,30S, 33S,36S)-N-(2-amino-2-oxo-ethyl)-36- [(4-chlorophenyl)methyl]-33-ethyl- 9,18-bis(3-guanidinopropyl)-21- (hydroxymethyl)-15-[(4- hydroxyphenyl)methyl]-12,27- bis(1H-imidazol-4-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1739.75 |
| 23 | 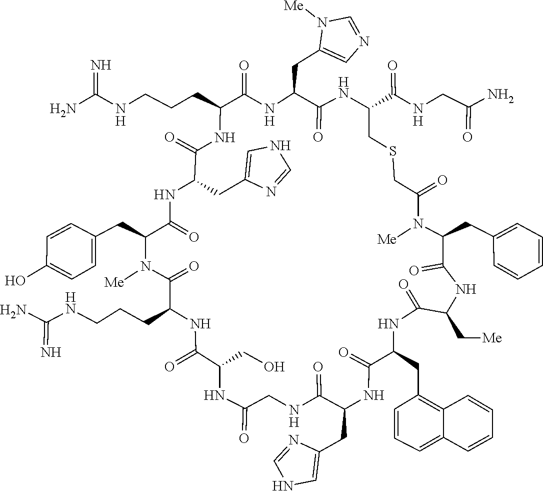 [SMILES: CC[C@@H]1NC(=O)[C@H](Cc2ccccc2)N(C)C(=O)CSC[C@@H](C(=O)NCC(N)=O)NC(=O)[C@H](Cc2cncn2C)NC(=O)[C@H](CCCNC(=N)N)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | (3R,6S,9S,12S,15S,18S,21S,27S,30S, 33S,36S)-N-(2-amino-2-oxo-ethyl)-36- benzyl-33-ethyl-9,18-bis(3- guanidinopropyl)-21- (hydroxymethyl)-15-[(4- hydroxyphenyl)methyl]-12,27- bis(1H-imidazol-4-ylmethyl)-16,37- dimethyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1719.80 |
| 24 | 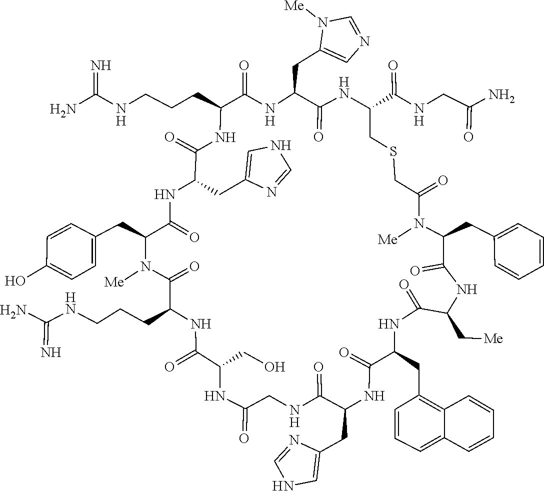 [SMILES: CC[C@@H]1NC(=O)[C@H](Cc2ccccc2)N(C)C(=O)CSC[C@@H](C(=O)NCC(N)=O)NC(=O)[C@H](Cc2cncn2C)NC(=O)[C@H](CCCNC(=N)N)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | (3R,6S,9S,12S,15S,18S,21S,27S,30S, 33S,36S)-N-(2-amino-2-oxo-ethyl)-36- benzyl-33-ethyl-9,18-bis(3- guanidinopropyl)-21- (hydroxymethyl)-15-[(4- hydroxyphenyl)methyl]-12,27- bis(1H-imidazol-4-ylmethyl)-16- methyl-6-[(3-methylimidazol-4- yl)methyl]- 5,8,11,14,17,20,23,26,29,32,35,38- dodecaoxo-30-[(4- phenylphenyl)methyl]-1-thia- 4,7,10,13,16,19,22,25,28,31,34,37- dodecazacyclononatriacontane-3- carboxamide | 1731.80 |
| 25 | 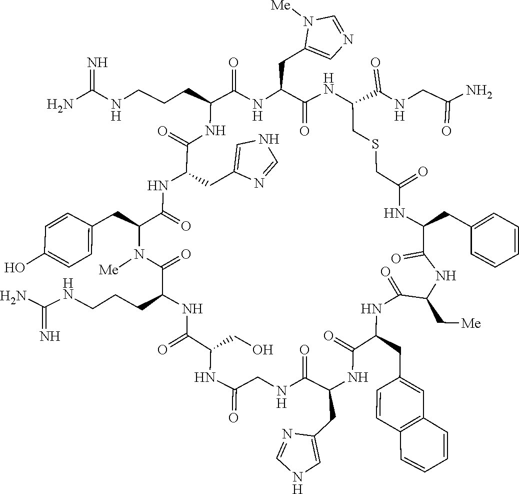 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2ccc3ccccc3c2)NC1=O] | (3R,6S,9S,12S,15S,18S,21S,27S,30S,3 3S,36S)-N-(2-amino-2-oxo-ethyl)-36- benzyl-33-ethyl-9,18-bis(3- guanidinopropyl)-21- (hydroxymethyl)-15-[(4- hydroxyphenyl)methyl]-12,27- bis(1H-imidazol-4-ylmethyl)-16- methyl-6-[(3-methylimidazol-4- yl)methyl]-30-(2-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1705.78 |
| 26 | 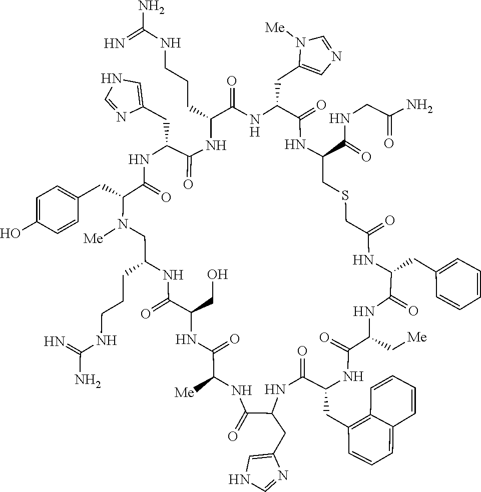 [SMILES: CC[C@H]1NC(=O)[C@@H](Cc2ccccc2)NC(=O)CSC[C@H](C(=O)NCC(N)=O)NC(=O)[C@@H](Cc2cncn2C)NC(=O)[C@@H](CCCNC(=N)N)NC(=O)[C@@H](Cc2c[nH]cn2)NC(=O)[C@@H](Cc2ccc(O)cc2)N(C)C[C@@H](CCCNC(=N)N)NC(=O)[C@@H](CO)NC(=O)[C@H](C)NC(=O)C(Cc2c[nH]cn2)NC(=O)[C@@H](Cc2cccc3ccccc23)NC1=O] | (3R,6S,9S,12S,15S,18S,21S,24R,27S, 30S,33S,36S)-N-(2-amino-2-oxo- ethyl)-36-benzyl-33-ethyl-9,18-bis(3- guanidinopropyl)-21- (hydroxymethyl)-15-[(4- hydroxyphenyl)methyl]-12,27- bis(1H-imidazol-4-ylmethyl)-16,24- dimethyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1719.80 |
| 27 | 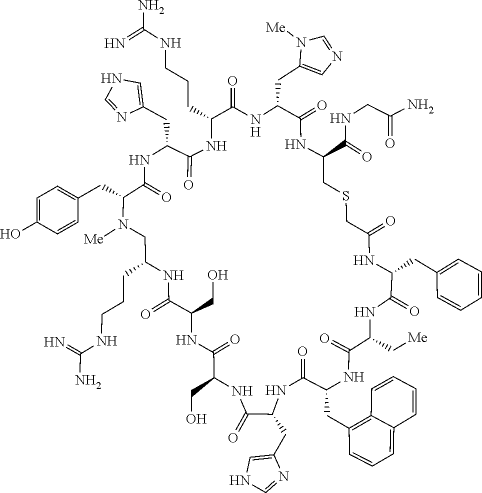 [SMILES: CC[C@H]1NC(=O)[C@@H](Cc2ccccc2)NC(=O)CSC[C@H](C(=O)NCC(N)=O)NC(=O)[C@@H](Cc2cncn2C)NC(=O)[C@@H](CCCNC(=N)N)NC(=O)[C@@H](Cc2c[nH]cn2)NC(=O)[C@@H](Cc2ccc(O)cc2)N(C)C[C@@H](CCCNC(=N)N)NC(=O)[C@@H](CO)NC(=O)[C@H](CO)NC(=O)[C@@H](Cc2c[nH]cn2)NC(=O)[C@@H](Cc2cccc3ccccc23)NC1=O] | (3R,6S,9S,12S,15S,18S,21S,24R,27S, 30S,33S,36S)-N-(2-amino-2-oxo- ethyl)-36-benzyl-33-ethyl-9,18-bis(3- guanidinopropyl)-21,24- bis(hydroxymethyl)-15-[(4- hydroxyphenyl)methyl]-12,27- bis(1H-imidazol-4-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1735.79 |
| 28 | 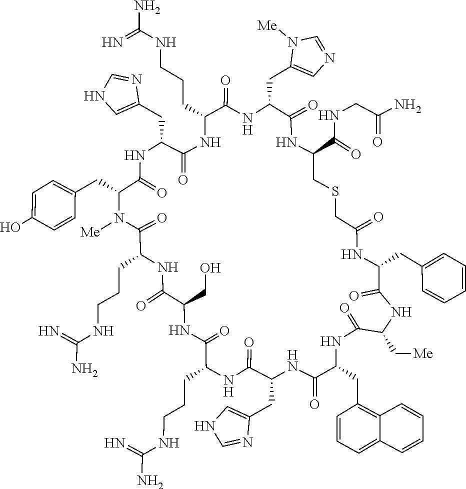 [SMILES: CC[C@H]1NC(=O)[C@@H](Cc2ccccc2)NC(=O)CSC[C@H](C(=O)NCC(N)=O)NC(=O)[C@@H](Cc2cncn2C)NC(=O)[C@@H](CCCNC(=N)N)NC(=O)[C@@H](Cc2c[nH]cn2)NC(=O)[C@@H](Cc2ccc(O)cc2)N(C)C(=O)[C@@H](CCCNC(=N)N)NC(=O)[C@@H](CO)NC(=O)[C@@H](CCCNC(=N)N)NC(=O)[C@@H](Cc2c[nH]cn2)NC(=O)[C@@H](Cc2cccc3ccccc23)NC1=O] | (3R,6S,9S,12S,15S,18S,21S,24S,27S,3 OS,33S,36S)-N-(2-amino-2-oxo- ethyl)-36-benzyl-33-ethyl-9,18,24- tris(3-guanidinopropyl)-21- (hydroxymethyl)-15-[(4- hydroxyphenyl)methyl]-12,27- bis(1H-imidazol-4-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1804.86 |
| 29 | 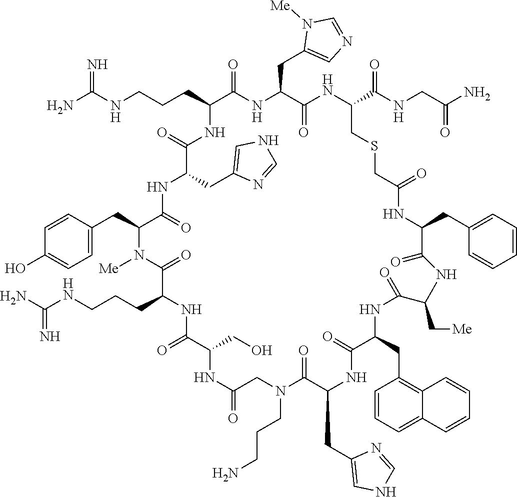 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CN(CCCN)C(=O)[C@H](Cc2c[nH]cn2)NC(=O)[C@H](Cc2cccc3ccccc23)NC1=O] | (3R,6S,9S,12S,15S,18S,21S,27S,30S,3 3S,36S)-N-(2-amino-2-oxo-ethyl)-25- (3-aminopropyl)-36-benzyl-33-ethyl- 9,18-bis(3-guanidinopropyl)-21- (hydroxymethyl)-15-[(4- hydroxyphenyl)methyl]-12,27- bis(1H-imidazol-4-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1762.84 |
| 30 | 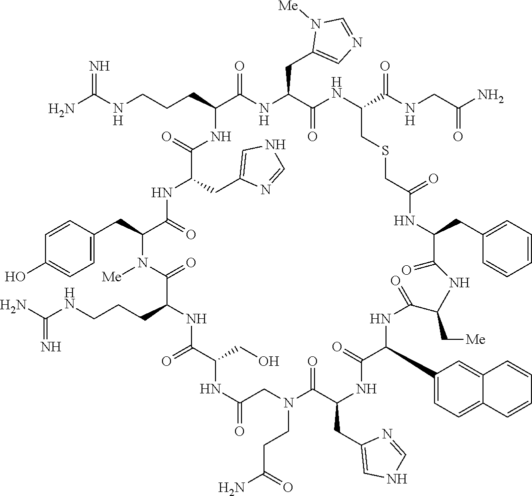 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CN(CCC(N)=O)C(=O)[C@H](Cc2c[nH]cn2)NC(=O)[C@H](c2ccc3ccccc3c2)NC1=O] | (3R,6S,9S,12S,15S,18S,21S,27S,30S, 33S,36S)-N-(2-amino-2-oxo-ethyl)-25- (3-amino-3-oxo-propyl)-36-benzyl- 33-ethyl-9,18-bis(3- guanidinopropyl)-21- (hydroxymethyl)-15-[(4- hydroxyphenyl)methyl]-12,27- bis(1H-imidazol-4-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1776.82 |
| 31 | 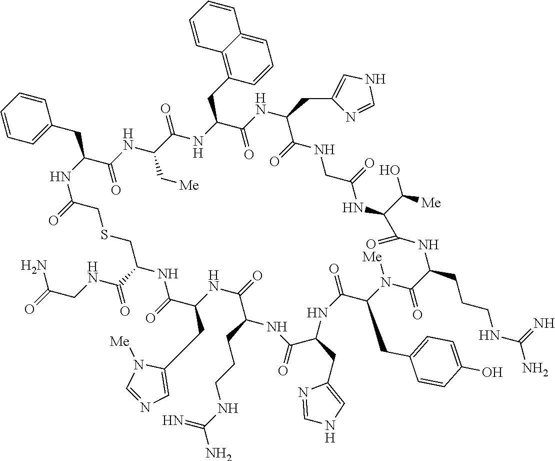 [SMILES: CC[C@@H]1NC(=O)[C@H](Cc2ccccc2)NC(=O)CSC[C@@H](C(=O)NCC(N)=O)NC(=O)[C@H](CCc2cncn2C)NC(=O)[C@H](CCCNC(=N)N)NC(=O)[C@H](Cc2c[nH]cn2)NC(=O)[C@H](Cc2ccc(O)cc2)N(C)C(=O)[C@H](CCCNC(=N)N)NC(=O)[C@H]([C@H](C)O)NC(=O)CNC(=O)[C@H](Cc2c[nH]cn2)NC(=O)[C@H](Cc2cccc3ccccc23)NC1=O] | (3R,6S,9S,12S,15S,18S,21S,27S,30S, 33S,36S)-N-(2-amino-2-oxo-ethyl)-36- benzyl-33-ethyl-9,18-bis(3- guanidinopropyl)-21-[(1S)-1- hydroxyethyl]-15-[(4- hydroxyphenyl)methyl]-12,27- bis(1H-imidazol-4-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1719.80 |
| 32 | 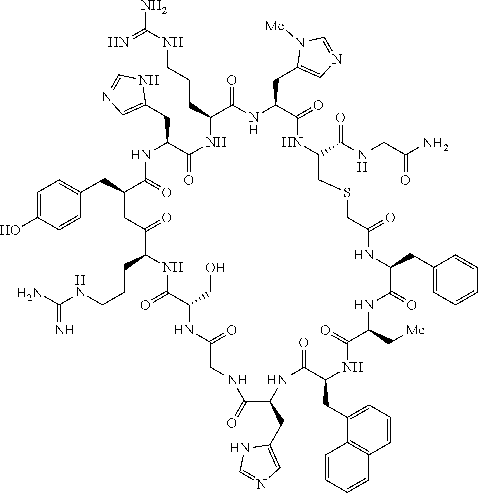 | (3R,6S,9S,12S,15S,18S,21S,27S,30S, 33S,36S)-N-(2-amino-2-oxo-ethyl)-36- benzyl-33-ethyl-9,18-bis(3- guanidinopropyl)-21- (hydroxymethyl)-15-[(4- hydroxyphenyl)methyl]-12,27- bis(1H-imidazol-4-ylmethyl)-6-[(3- methylimidazol-4-yl)methyl]-30-(1- naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1691.77 |
| 33 | 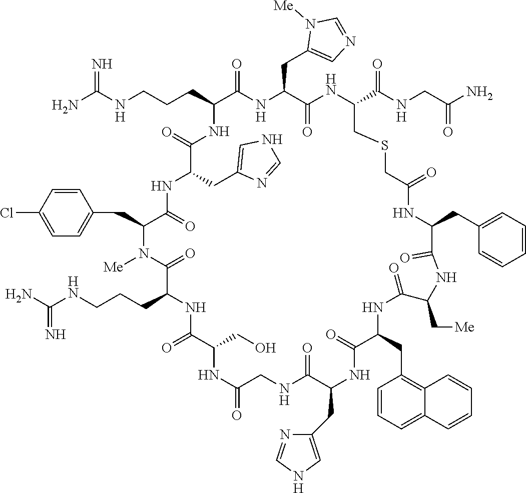 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)NC(=O)[C@H](Cc2c[nH]cn2)NC(=O)[C@H](Cc2ccc(Cl)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | (3R,6S,9S,12S,15S,18S,21S,27S,30S, 33S,36S)-N-(2-amino-2-oxo-ethyl)-36- benzyl-15-[(4-chlorophenyl)methyl]- 33-ethyl-9,18-bis(3- guanidinopropyl)-21- (hydroxymethyl)-12,27-bis(1H- imidazol-4-ylmethyl)-16-methyl-6- [(3-methylimidazol-4-yl)methyl]-30- (1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1723.75 |
| 34 | 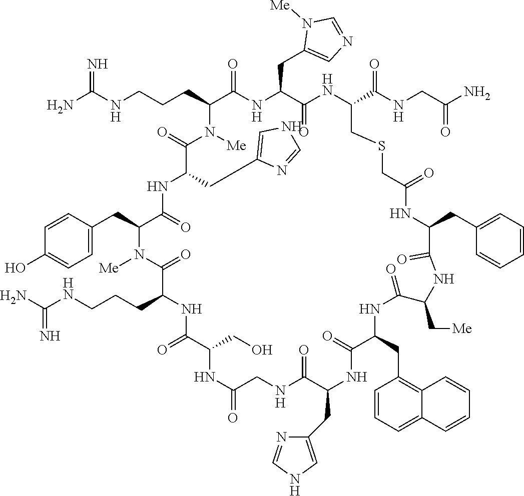 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)N(C)C(=O)[C@H](CCc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | (3R,6S,9S,12S,15S,18S,21S,27S,30S, 33S,36S)-N-(2-amino-2-oxo-ethyl)-36- benzyl-33-ethyl-9,18-bis(3- guanidinopropyl)-21- (hydroxymethyl)-15-[(4- hydroxyphenyl)methyl]-12,27- bis(1H-imidazol-4-ylmethyl)-10,16- dimethyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1719.80 |
| 35 | 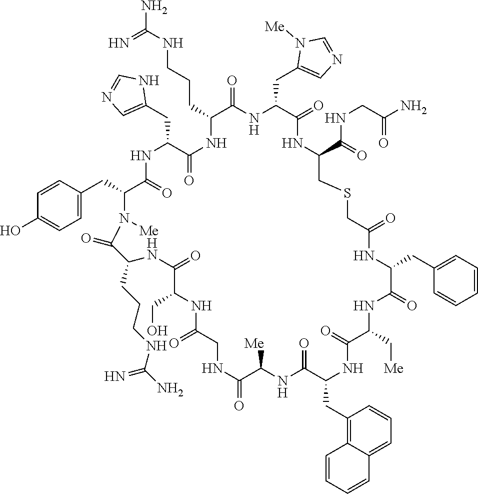 [SMILES: CC[C@H]1NC(=O)[C@@H](Cc2ccccc2)NC(=O)CSC[C@H](C(=O)NCC(N)=O)NC(=O)[C@@H](Cc2cncn2C)NC(=O)[C@@H](CCCNC(=N)N)NC(=O)[C@@H](Cc2cnc[nH]2)NC(=O)[C@@H](Cc2ccc(O)cc2)N(C)C(=O)[C@@H](CCCNC(=N)N)NC(=O)[C@@H](CO)NC(=O)CNC(=O)[C@@H](C)NC(=O)[C@@H](Cc2cccc3ccccc23)NC1=O] | (3R,6S,9S,12S,15S,18S,21S,27S,30S, 33S,36S)-N-(2-amino-2-oxo-ethyl)-36- benzyl-33-ethyl-9,18-bis(3- guanidinopropyl)-21- (hydroxymethyl)-15-[(4- hydroxyphenyl)methyl]-12-(1H- imidazol-4-ylmethyl)-16,27- dimethyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1639.76 |
| 36 | 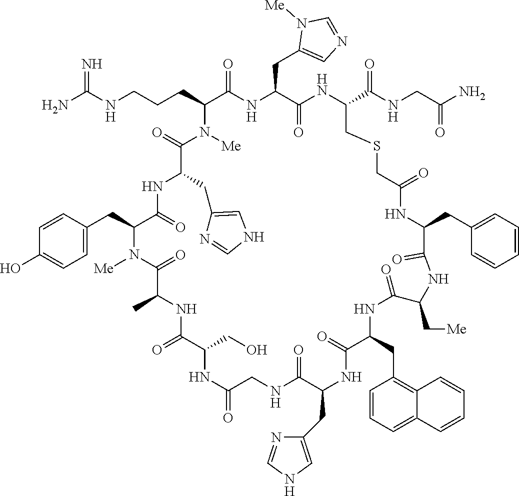 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)N(C)C(=O)[C@H](Cc2c[nH]cn2)NC(=O)[C@H](Cc2ccc(O)cc2)N(C)C(=O)[C@H](C)NC(=O)[C@H](CO)NC(=O)CNC(=O)[C@H](Cc2c[nH]cn2)NC(=O)[C@H](Cc2cccc3ccccc23)NC1=O] | (3R,6S,9S,12S,15S,18S,21S,27S,30S, 33S,36S)-N-(2-amino-2-oxo-ethyl)-36- benzyl-33-ethyl-9-(3- guanidinopropyl)-21- (hydroxymethyl)-15-[(4- hydroxyphenyl)methyl]-12,27- bis(1H-imidazol-4-ylmethyl)-16,18- dimethyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1620.72 |
| 37 | 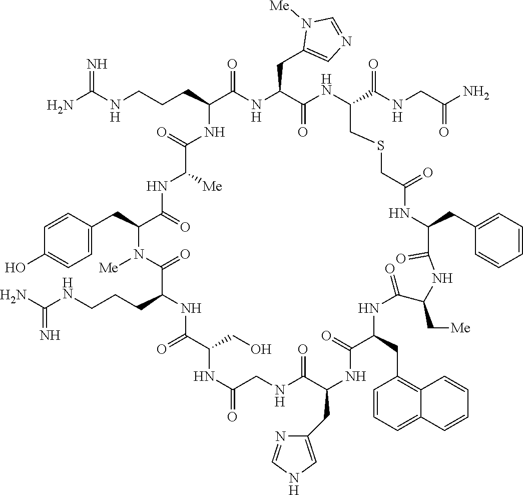 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)NC(=O)[C@H](C)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | (3R,6S,9S,12S,15S,18S,21S,27S,30S, 33S,36S)-N-(2-amino-2-oxo-ethyl)-36- benzyl-33-ethyl-9,18-bis(3- guanidinopropyl)-21- (hydroxymethyl)-15-[(4- hydroxyphenyl)methyl]-27-(1H- imidazol-4-ylmethyl)-12,16- dimethyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1639.76 |
| 38 | 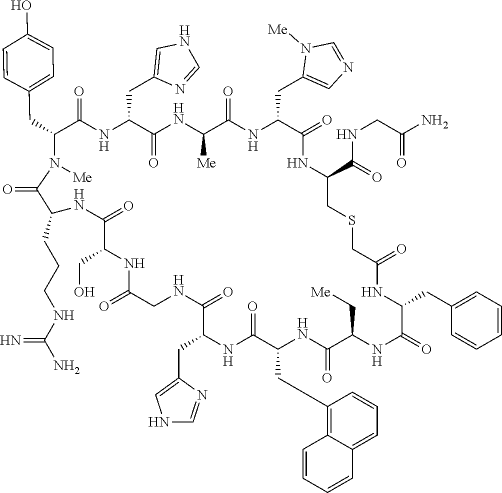 [SMILES: CC[C@H]1NC(=O)[C@@H](Cc2ccccc2)NC(=O)CSC[C@H](C(=O)NCC(N)=O)NC(=O)[C@@H](Cc2cncn2C)NC(=O)[C@@H](C)NC(=O)[C@@H](Cc2c[nH]cn2)NC(=O)[C@@H](Cc2ccc(O)cc2)N(C)C(=O)[C@@H](CCCNC(=N)N)NC(=O)[C@@H](CO)NC(=O)CNC(=O)[C@@H](Cc2c[nH]cn2)NC(=O)[C@@H](Cc2cccc3ccccc23)NC1=O] | (3R,6S,9S,12S,15S,18S,21S,27S,30S, 33S,36S)-N-(2-amino-2-oxo-ethyl)-36- benzyl-33-ethyl-18-(3- guanidinopropyl)-21- (hydroxymethyl)-15-[(4- hydroxyphenyl)methyl]-12,27- bis(1H-imidazol-4-ylmethyl)-9,16- dimethyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1620.72 |
| 39 | 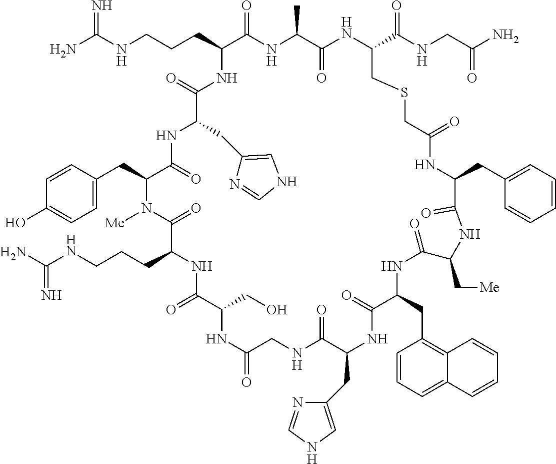 [SMILES: CC[C@@H]1NC(=O)[C@H](Cc2ccccc2)NC(=O)CSC[C@@H](C(=O)NCC(N)=O)NC(=O)[C@H](C)NC(=O)[C@H](CCCNC(=N)N)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | (3R,6S,9S,12S,15S,18S,21S,27S,30S, 33S,36S)-N-(2-amino-2-oxo-ethyl)-36- benzyl-33-ethyl-9,18-bis(3- guanidinopropyl)-21- (hydroxymethyl)-15-[(4- hydroxyphenyl)methyl]-12,27- bis(1H-imidazol-4-ylmethyl)-6,16- dimethyl-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1625.75 |
| 40 | 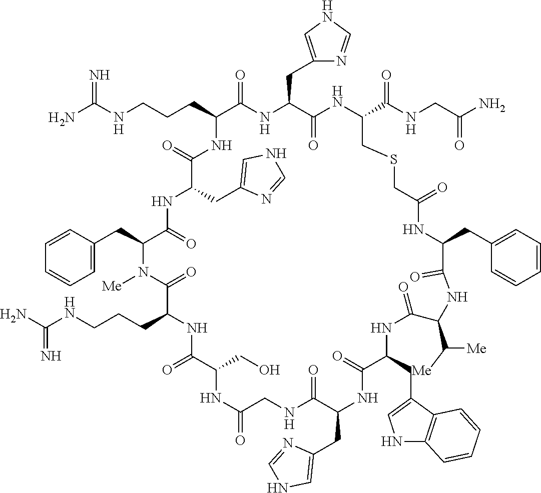 [SMILES: CC(C)[C@@H]1NC(=O)[C@H](Cc2ccccc2)NC(=O)CSC[C@@H](C(=O)NCC(N)=O)NC(=O)[C@H](Cc2c[nH]cn2)NC(=O)[C@H](CCCNC(=N)N)NC(=O)[C@H](Cc2c[nH]cn2)NC(=O)[C@H](Cc2ccccc2)N(C)C(=O)[C@H](CCCNC(=N)N)NC(=O)[C@H](CO)NC(=O)CNC(=O)[C@H](Cc2c[nH]cn2)NC(=O)[C@H](Cc2c[nH]c3ccccc23)NC1=O] | (3R,6S,9S,12S,15S,18S,21S,27S,30S,3 3S,36S)-N-(2-amino-2-oxo-ethyl)- 15,36-dibenzyl-9,18-bis(3- guanidinopropyl)-21- (hydroxymethyl)-6,12,27-tris(1H- imidazol-4-ylmethyl)-30-(1H-indol-3- ylmethyl)-33-isopropyl- 5,8,11,14,17,20,23,26,29,32,35,38- dodecaoxo-1-thia- 4,7,10,13,16,19,22,25,28,31,34,37- dodecazacyclononatriacontane-3- carboxamide | 1664.77 |
| 41 | 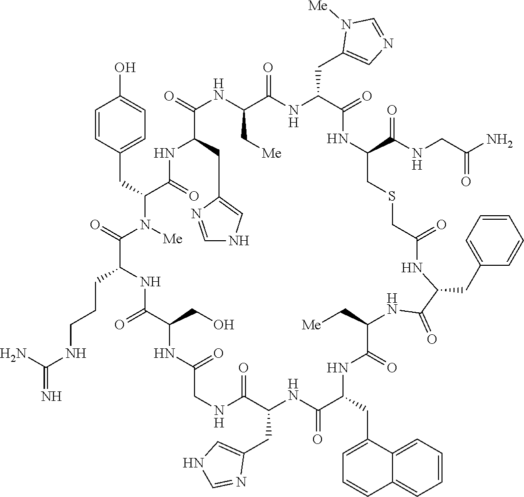 [SMILES: CC[C@H]1NC(=O)[C@@H](Cc2ccccc2)NC(=O)CSC[C@H](C(=O)NCC(N)=O)NC(=O)[C@@H](Cc2cncn2C)NC(=O)[C@@H](CC)NC(=O)[C@@H](Cc2c[nH]cn2)NC(=O)[C@@H](Cc2ccc(O)cc2)N(C)C(=O)[C@@H](CCCNC(=N)N)NC(=O)[C@@H](CO)NC(=O)CNC(=O)[C@@H](Cc2c[nH]cn2)NC(=O)[C@@H](Cc2cccc3ccccc23)NC1=O] | (3R,6S,9S,12S,15S,18S,21S,27S,30S, 33S,36S)-N-(2-amino-2-oxo-ethyl)-36- benzyl-9,33-diethyl-18-(3- guanidinopropyl)-21- (hydroxymethyl)-15-[(4- hydroxyphenyl)methyl]-12,27- bis(1H-imidazol-4-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1634.74 |
| 42 | 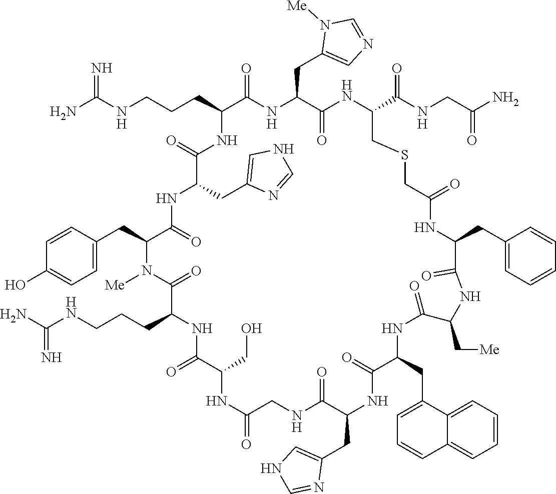 [SMILES: CC[C@@H]1NC(=O)[C@H](Cc2ccccc2)NC(=O)CSC[C@@H](C(=O)NCC(N)=O)NC(=O)[C@H](Cc2cncn2C)NC(=O)[C@H](CCCNC(=N)N)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | rac- (3R,6S,9S,12S,15S,18S,21S,27S,30S, 33S,36S)-N-(2-amino-2-oxo-ethyl)-9- (3-amino-3-oxo-propyl)-36-benzyl- 33-ethyl-18-(3-guanidinopropyl)-21- (hydroxymethyl)-15-[(4- hydroxyphenyl)methyl]-12,27- bis(1H-imidazol-4-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1677.74 |
| 43 | 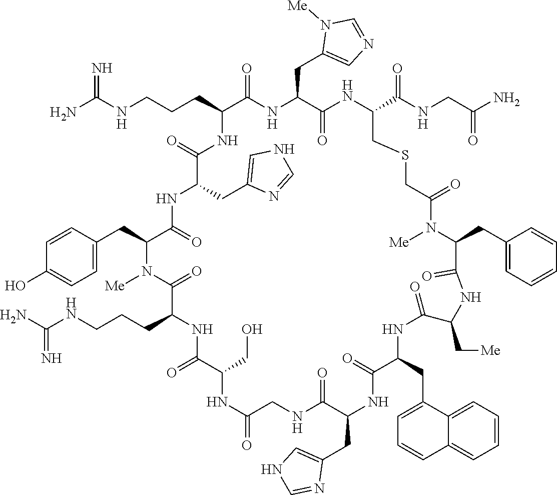 [SMILES: CC[C@@H]1NC(=O)[C@H](Cc2ccccc2)N(C)C(=O)CSC[C@@H](C(=O)NCC(N)=O)NC(=O)[C@H](Cc2cncn2C)NC(=O)[C@H](CCCNC(=N)N)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | ac- (3R,6S,9S,12S,15S,18S,21S,27S,30S, 33S,36S)-N-(2-amino-2-oxo-ethyl)-36- benzyl-33-ethyl-9,18-bis(3- guanidinopropyl)-21- (hydroxymethyl)-15-[(4- hydroxyphenyl)methyl]-12,27- bis(1H-imidazol-4-ylmethyl)- 10,16,37-trimethyl-6-[(3- methylimidazol-4-yl)methyl]-30-(1- naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1733.82 |
| 44 | 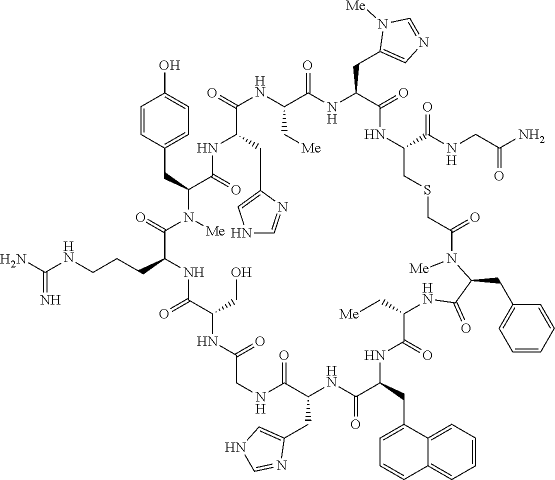 [SMILES: CC[C@@H]1NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@@H](Cc2c[nH]cn2)NC(=O)[C@H](Cc2cccc3ccccc23)NC(=O)[C@H](CC)NC(=O)[C@H](Cc2ccccc2)N(C)C(=O)CSC[C@@H](C(=O)NCC(N)=O)NC(=O)[C@H](Cc2cncn2C)NC1=O] | rac- (3R,6S,9S,12S,15S,18S,21S,27S,30S, 33S,36S)-N-(2-amino-2-oxo-ethyl)-36- benzyl-9,33-diethyl-18-(3- guanidinopropyl)-21- (hydroxymethyl)-15-[(4- hydroxyphenyl)methyl]-12,27- bis(1H-imidazol-4-ylmethyl)-16,37- dimethyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1648.75 |
| 45 | 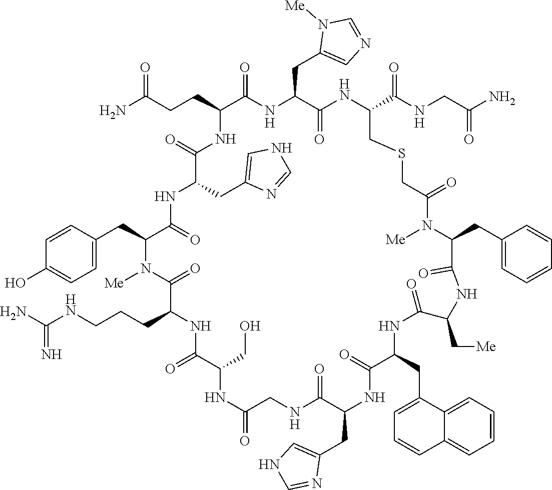 [SMILES: CC[C@@H]1NC(=O)[C@H](Cc2ccccc2)N(C)C(=O)CSC[C@@H](C(=O)NCC(N)=O)NC(=O)[C@H](Cc2cncn2C)NC(=O)[C@H](CCC(N)=O)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | rac- (3R,6S,9S,12S,15S,18S,21S,27S,30S, 33S,36S)-N-(2-amino-2-oxo-ethyl)-9- (3-amino-3-oxo-propyl)-36-benzyl- 33-ethyl-18-(3-guanidinopropyl)-21- (hydroxymethyl)-15-[(4- hydroxyphenyl)methyl]-12,27- bis(1H-imidazol-4-ylmethyl)-16,37- dimethyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1691.76 |
| 46 | 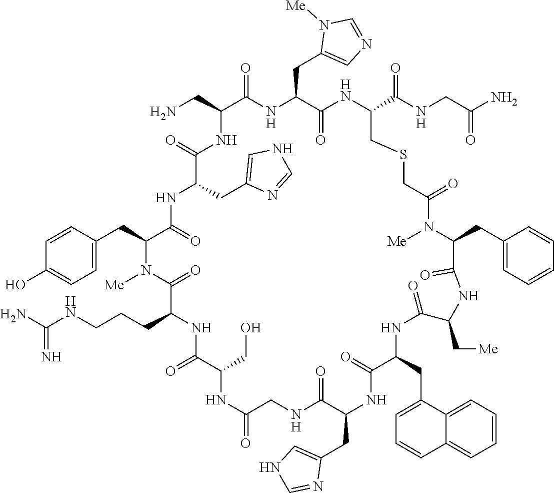 [SMILES: CC[C@@H]1NC(=O)[C@H](Cc2ccccc2)N(C)C(=O)CSC[C@@H](C(=O)NCC(N)=O)NC(=O)[C@H](Cc2cncn2C)NC(=O)[C@H](CN)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | rac- (3R,6S,9S,12S,15S,18S,21S,27S,30S, 33S,36S)-9-(aminomethyl)-N-(2- amino-2-oxo-ethyl)-36-benzyl-33- ethyl-18-(3-guanidinopropyl)-21- (hydroxymethyl)-15-[(4- hydroxyphenyl)methyl]-12,27- bis(1H-imidazol-4-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1635.73 |
| 47 | 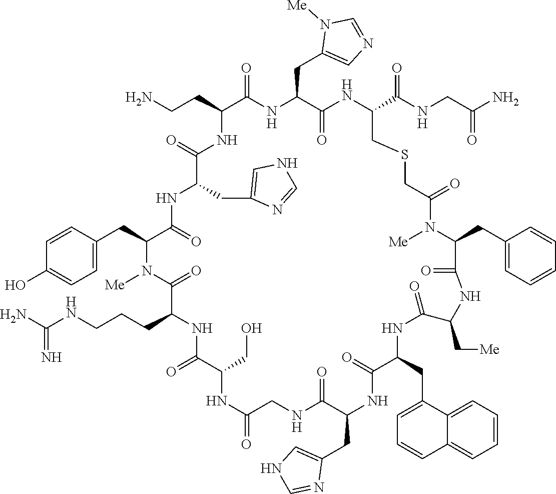 [SMILES: CC[C@@H]1NC(=O)[C@H](Cc2ccccc2)N(C)C(=O)CSC[C@@H](C(=O)NCC(N)=O)NC(=O)[C@H](Cc2cncn2C)NC(=O)[C@H](CCN)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | rac- (3R,6S,9S,12S,15S,18S,21S,27S,30S, 33S,36S)-9-(2-aminoethyl)-N-(2- amino-2-oxo-ethyl)-36-benzyl-33- ethyl-18-(3-guanidinopropyl)-21- (hydroxymethyl)-15-[(4- hydroxyphenyl)methyl]-12,27- bis(1H-imidazol-4-ylmethyl)-16- methyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1635.73 |
| 48 | 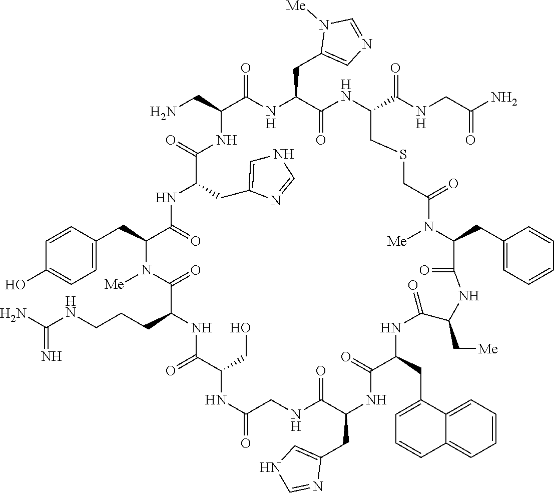 [SMILES: CC[C@@H]1NC(=O)[C@H](Cc2ccccc2)N(C)C(=O)CSC[C@@H](C(=O)NCC(N)=O)NC(=O)[C@H](Cc2cncn2C)NC(=O)[C@H](CN)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | rac- (3R,6S,9S,12S,15S,18S,21S,27S,30S,3 3S,36S)-9-(aminomethyl)-N-(2- amino-2-oxo-ethyl)-36-benzyl-33- ethyl-18-(3-guanidinopropyl)-21- (hydroxymethyl)-15-[(4- hydroxyphenyl)methyl]-12,27- bis(1H-imidazol-4-ylmethyl)-16,37- dimethyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1649.75 |
| 49 | 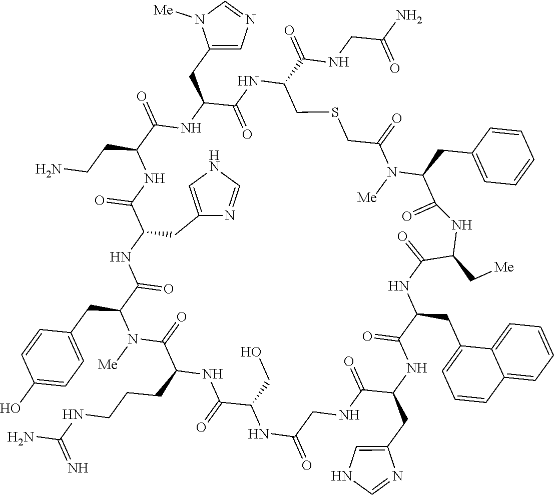 [SMILES: CC[C@@H]1NC(=O)[C@H](Cc2ccccc2)N(C)C(=O)CSC[C@@H](C(=O)NCC(N)=O)NC(=O)[C@H](Cc2cncn2C)NC(=O)[C@H](CCN)NC(=O)[C@H](Cc2c[nH]cn2)NC(=O)[C@H](Cc2ccc(O)cc2)N(C)C(=O)[C@H](CCCNC(=N)N)NC(=O)[C@H](CO)NC(=O)CNC(=O)[C@H](Cc2c[nH]cn2)NC(=O)[C@H](Cc2cccc3ccccc23)NC1=O] | rac- (3R,6S,9S,12S,15S,18S,21S,27S,30S, 33S,36S)-9-(2-aminoethyl)-N-(2- amino-2-oxo-ethyl)-36-benzyl-33- ethyl-18-(3-guanidinopropyl)-21- (hydroxymethyl)-15-[(4- hydroxyphenyl)methyl]-12,27- bis(1H-imidazol-4-ylmethyl)-16,37- dimethyl-6-[(3-methylimidazol-4- yl)methyl]-30-(1-naphthylmethyl)- 5,8,11,14,17,20,23,26,29,32,35,38- dodecaoxo-1-thia- 4,7,10,13,16,19,22,25,28,31,34,37- dodecazacyclononatriacontane-3- carboxamide | 1663.76 |

In another embodiment of the invention, there is a cyclic dodecapeptide of Formula (Ia), or a pharmaceutically acceptable salt, solvate, or stereoisomer thereof:

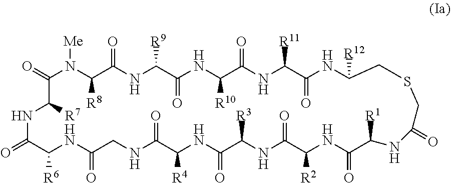

In some embodiments there is a compound of formula (Ia) wherein in R¹, R², R³, R⁴, R⁵, R⁶, R⁷, R⁸, R⁹, R¹⁰, R¹¹ and R¹² are as described above.

In one embodiment of the present invention there is a compound of formula (Ib) wherein:

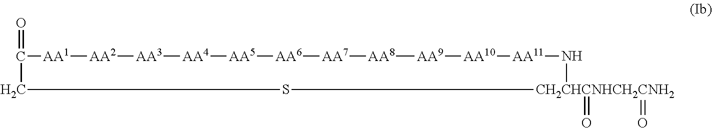

AA¹ is an aromatic amino acid or N-methyl-aromatic amino acid wherein the aromatic amino acid is phenylalanine wherein the phenyl ring of either is optionally substituted by 1 to 3 C₁₋₆ alkyl, halogens, C₁₋₆-haloalkyl, hydroxyl, C₁₋₆-alkoxy, C₁₋₆-haloalkoxy, C₁₋₆-thioalkyl, C₁₋₆-alkylsulfonyl or cyano;
AA² is an aliphatic amino acid of formula H₂NCHR^{a}CO₂H wherein R^{a} is C₁₋₆alkyl or C₁₋₃alkoxy-C₁₋₃ alkyl;
AA³ is an aromatic amino acid of formula H₂NCHR^{a}CO₂H wherein R^{a} is 1-naphthylmethyl, 2-naphthylmethyl or 4-phenylbenzyl and R^{a} is optionally substituted by 1 to 3 C₁₋₆ alkyl, halogens, C₁₋₆-haloalkyl, hydroxyl, C₁₋₆-alkoxy, C₁₋₆-haloalkoxy, C₁₋₆-thioalkyl, C₁₋₆-alkylsulfonyl or cyano, or a heteroarylamino acid wherein R^{a} is 3-indolemethyl optionally C₁₋₃ N-alkyl-3-indolemethyl;
AA⁴ is histidine;
AA⁵ is an aliphatic amino acid wherein said aliphatic amino acid is a compound of formula H₂NCHR^{a}CO₂H wherein R^{a} is hydrogen, methyl or hydroxymethyl, preferably, AA⁵ is glycine;
AA⁶ is arginine an amino acid of formula H₂NCHR^{a}CO₂H wherein R^{a} is C₁₋₆ hydroxyalkyl, preferably AA⁷ is serine;
AA⁷ is a basic amino acid f formula H₂NCHR^{a}CO₂H wherein R^{a} is guanidino-C₁₋₄-alkyl, preferably AA⁸ is arginine
AA⁸ is an aromatic amino acid or N-methyl-aromatic amino acid wherein the aromatic amino acid is phenylalanine, optionally substituted by 1 to three C₁₋₆ alkyl, halogens, C₁₋₆-haloalkyl, C₁₋₆-alkoxy, C₁₋₆-haloalkoxy, C₁₋₆-thioalkyl; preferably AA⁹ is phenylalanine;
AA⁹ is a heteroaryl amino acid wherein said heteroaryl amino acid is a compound of formula H₂NCHR^{a}CO₂H wherein R^{a} is 1H-imidazole-5-ylmethyl, 3-methylimidazol-4-yl-methyl, preferably AA¹⁰ is histidine;
AA¹⁰ is a basic amino acid wherein said basic amino acid is a compound of formula H₂NCHR^{a}CO₂H wherein R^{a} is C₁₋₆aminoalkyl, guanidino-C₁₋₄-alkyl, (CH₂)₁₋₃CONH₂, or C₁₋₆ alkyl;
AA¹¹ is a heteroaryl amino acid wherein said heteroaryl amino acid is a compound of formula H₂NCHR^{a}CO₂H wherein R^{a} is 1H-imidazole-5-ylmethyl, 3-methylimidazol-4-yl-methyl, preferably R^{a} is 3-methylimidazol-4-yl-methyl.

In another embodiment there is afforded a compound of formula (Ib) wherein:
AA¹ is phenylalanine; AA² is an aliphatic amino acid of formula H₂NCHR^{a}CO₂H wherein R^{a} is ethyl, isopropyl or sec-butyl; AA³ is 2-amino-3-(naphthalen-1-yl)propanoic acid or tryptophan; AA⁴ is histidine; AA⁵ is glycine; AA⁶ is aliphatic amino acid of formula H₂NCHR^{a}CO₂H wherein R^{a} is hydroxylmethyl or —CH(OH)Me; AA⁷ is arginine; AA⁸ is phenylalanine optionally substituted with hydroxyl or halo; AA⁹ is histidine; AA¹⁰ is arginine, amino methyl, aminoethyl or amino butyl; AA¹¹ is a basic amino acid of formula H₂NCHR^{a}CO₂H wherein R^{a} is 3-methylimidazol-4-yl)methyl.

In other embodiments of the invention, a cyclic tetradecapeptide of Formula (II), or a pharmaceutically acceptable salt, solvate, or stereoisomer thereof as disclosed above in the Summary of the Disclosure.

In one embodiment of a compound of Formula (II), R^{1a} is methyl or hydrogen. In one embodiment of a compound of Formula (II), R^{1a} is hydrogen. In another embodiment of a compound of Formula (II), R^{1a} is methyl.

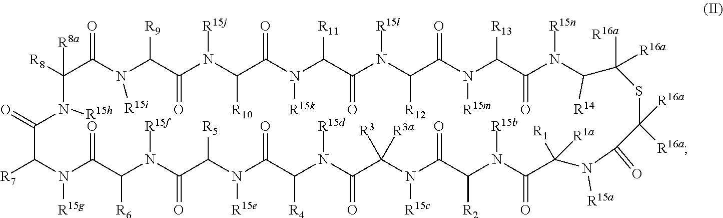

In some embodiments of a compound of Formula (II), R¹ is optionally substituted benzyl. In some embodiments of a compound of Formula (II), R¹ is unsubstituted benzyl.

In some embodiments of a compound of Formula (II), R² is 4-(imidazolyl)methyl optionally substituted with C₁₋₆alkyl. In some embodiments of a compound of Formula (II), R² is 4-(imidazolyl)methyl with a N-substituted methyl. In some embodiments of a compound of Formula (II), 3-(methylimidazol-4-yl)methyl (iib wherein R^{b} is methyl). In some embodiments of a compound of Formula (II), R² is 4-(imidazolyl)methyl.

In some embodiments of a compound of formula (II) R³ is C₁₋₆alkyl. In some embodiments of a compound of Formula (II), R³ is (1S)-methylpropyl. In some embodiments of a compound of Formula (II), in some embodiments R³ is methyl. In some embodiments of a compound of Formula (II), R³ is C₁₋₆alkyl hydroxyalkyl. In some embodiments of a compound of Formula (II), R³ is (1R)-1-hydroxyethyl. In some embodiments of a compound of formula (II) R⁴ is C₁₋₆ alkyl; in some embodiments R⁴ is (1S)-methylpropyl; in some embodiments R⁴ is methyl.

In some embodiments of a compound of formula (II), R⁵ is 4-(imidazolyl)methyl optionally substituted with C₁₋₆alkyl. n some embodiments of a compound of formula (II), R⁵ is 4-(imidazolyl)methyl with a N-substituted methyl. n some embodiments of a compound of formula (II), R⁵ is 3-(methylimidazol-4-yl)methyl (iib wherein R^{b} is methyl). In some embodiments of a compound of formula (II), R⁵ is 4-(imidazolyl)methyl. In some embodiments of a compound of formula (II), R⁵ is optionally substituted 3-guanidino-C₂₋₄alkyl. In some embodiments of a compound of formula (II), R⁵ is 3-guanidinopropyl.

In some embodiments of a compound of formula (II), R⁶ is optionally substituted 3-guanido-C₂₋₄alkyl. In some embodiments of a compound of formula (II), R⁶ is 3-guanidopropyl. In some embodiments of a compound of formula (II), R⁶ is amino-C₁₋₆alkyl. In some embodiments of a compound of formula (II), R⁶ is 4-aminobutyl.

In some embodiments of a compound of formula (II), R⁷ is hydroxy-C₁₋₆alkyl. In some embodiments of a compound of formula (II), R⁷ is (1R)-1-hydroxyethyl. In some embodiments of a compound of formula (II), R⁷ is 3-guanido-C₂₋₄alkyl. In some embodiments of a compound of formula (II), R⁶ is 3-guanidopropyl.

In some embodiments of a compound of formula (II), R⁸ is optionally substituted benzyl. In some embodiments of a compound of formula (II), R⁸ is hydroxybenzyl. In some embodiments of a compound of formula (II), R⁸ is halo substituted benzyl. In some embodiments of a compound of formula (II), R⁸ is benzyl substituted by 1 or 2 halogens. In some embodiments of a compound of formula (II), R⁸ is benzyl substituted by 1 or 2 chlorines. In some embodiments of a compound of formula (II), R⁸ is 4-chlorobenzyl.

In some embodiments of a compound of formula (II), R⁹ is optionally substituted benzyl. In some embodiments of a compound of formula (II), R⁹ is hydroxybenzyl. In some embodiments of a compound of formula (II), R⁹ is 4-hydroxybenzyl. In some embodiments of a compound of formula (II), R⁹ is halo substituted benzyl. In some embodiments of a compound of formula (II), R⁹ is benzyl substituted by 1 or 2 halogens. In some embodiments of a compound of formula (II) in some embodiments R⁹ is benzyl substituted by 1 or 2 chlorines. In some embodiments of a compound of formula (II), R⁹ is 4-chlorobenzyl. In some embodiments of a compound of formula (II), R⁹ is unsubstituted benzyl.

In some embodiments of a compound of formula (II), R¹⁰ is optionally substituted 3-guanido-C₁₋₆alkyl. In some embodiments of a compound of formula (II), R¹⁰ is 3-guanidopropyl. In some embodiments of a compound of formula (II), R¹⁰ is amino-C₁₋₆alkyl. In some embodiments of a compound of formula (II), R¹⁰ is 4-aminobutyl. In some embodiments of a compound of formula (II), R¹⁰ is 4(imidazolyl)methyl optionally substituted with C₁₋₆alkyl. In some embodiments of a compound of formula (II), R¹⁰ is 4-(imidazolyl)methyl with a N-substituted methyl. In some embodiments of a compound of formula (II), R¹⁰ is 3-(methylimidazol-4-yl)methyl (iib wherein R^{b} is methyl). In some embodiments of a compound of formula (II), R¹⁰ is 4-(imidazolyl)methyl.

In some embodiments of a compound of formula (II), R¹¹ is optionally substituted benzyl. In some embodiments of a compound of formula (II), R¹¹ is hydroxybenzyl. In some embodiments of a compound of formula (II), R¹¹ is 4-hydroxybenzyl. In some embodiments of a compound of formula (II), R¹¹ is halo substituted benzyl. In some embodiments of a compound of formula (II), R¹¹ is benzyl substituted by 1 or 2 halogens. In some embodiments of a compound of formula (II), in some embodiments R¹¹ is benzyl substituted by 1 or 2 chlorines. In some embodiments of a compound of formula (II), R¹¹ is 4-chlorobenzyl. In some embodiments of a compound of formula (II), R¹¹ is unsubstituted benzyl.

In some embodiments of a compound of formula (II), R¹² is optionally substituted benzyl. In some embodiments of a compound of formula (II), R¹² is hydroxybenzyl. In some embodiments of a compound of formula (II), R¹² is 4-hydroxybenzyl. In some embodiments of a compound of formula (II), R¹² is halo substituted benzyl. In some embodiments of a compound of formula (II), R¹² is benzyl substituted by 1 or 2 halogens. In some embodiments of a compound of formula (II), R¹² is benzyl substituted by 1 or 2 chlorines. In some embodiments of a compound of formula (II), R¹² is 4-chlorobenzyl. In some embodiments of a compound of formula (II), R¹² is N-substituted-3-indolylmethyl wherein R^{f} is hydrogen or C₁₋₆alkyl. In some embodiments R¹² is N-substituted-3-indolylmethyl wherein R^{f} is hydrogen is hydrogen. In some embodiments R^{f} methyl. In some embodiments of a compound of formula (II), R¹² is indolyl-3-methyl (vi wherein R^{f} is hydrogen). In some embodiments of a compound of formula (II), R¹² is optionally substituted benzyl. In some embodiments of a compound of formula (II), R¹² is hydroxybenzyl. In some embodiments of a compound of formula (II), R¹² is 4-hydroxybenzyl. In some embodiments of a compound of formula (II), R¹² is halo substituted benzyl. In some embodiments of a compound of formula (II), R¹² is benzyl substituted by 1 or 2 halogens. In some embodiments of a compound of formula (II), R¹² is benzyl substituted by 1 or 2 chlorines. In some embodiments of a compound of formula (II), R¹² is 4-chlorobenzyl. In some embodiments of a compound of formula (II), R¹² is unsubstituted benzyl.

In some embodiments of a compound of formula (II), R¹³ is optionally substituted benzyl. In some embodiments of a compound of formula (II), R¹³ is hydroxybenzyl. In some embodiments of a compound of formula (II), R¹³ is 4-hydroxybenzyl. In some embodiments of a compound of formula (II), R¹³ is halo substituted benzyl. In some embodiments of a compound of formula (II), R¹³ is benzyl substituted by 1 or 2 halogens. In some embodiments of a compound of formula (II), R¹³ is benzyl substituted by 1 or 2 chlorines. In some embodiments of a compound of formula (II), R¹³ is 4-chlorobenzyl. In some embodiments of a compound of formula (II), R¹³ is unsubstituted benzyl.

In some embodiments of a compound of formula (II), R¹⁴ is N-(2-amino-2-oxo-ethyl)carboxamide.

In some embodiments of a compound of formula (II) R^{b}, R^{c}, R^{d}, R^{e}, R^{f}, R^{1a}, R^{3a}, R^{8a}, R^{15a}, R^{15b}, R^{15c}, R^{15d}R^{15e}, R^{15f}, R^{15g}, R¹⁵ⁱ, R^{15j}, R^{15k}, R^{16a}, R^{16b}, R^{16c} and R^{16d} are hydrogen or C₁₋₆ alkyl; in some embodiment R^{b}, R^{c}, R^{d}, R^{e}, R^{f}, R^{1a}, R^{3a}, R^{8a}, R^{15a}, R^{15b}, R^{15c}, R^{15d}, R^{15e}, R^{15f}, R^{15g}, R¹⁵ⁱ, R^{15j}, R^{15k}, R^{16a}, R^{16b}, R^{16c} and R^{16d} are hydrogen or methyl. In some embodiments of a compound of formula (II), R^{a} is hydrogen, C₁₋₆alkyl, hydroxyl or halo. In some embodiments of a compound of formula (II), R^{a} is chloro. In some embodiments of a compound of formula (II), R^{f} is hydrogen or C₁₋₆alkyl. In some embodiments of a compound of formula (II), R^{f} is hydrogen. In some embodiments of a compound of formula (II), R^{f} is methyl. In some embodiments of a compound of formula (II), n and p are independently from 0 to 2. i In some embodiments of a compound of formula (II), n and p are 1 or 0. In some embodiments of a compound of formula (II), n is 1. In some embodiments of a compound of formula (II), n is 0. In some embodiments of a compound of formula (II) q is independently from 0 or 1. In some embodiments of a compound of formula (II), q is 1. In some embodiments of a compound of formula (II), r is independently from 1 or 2. In some embodiments of a compound of formula (II), r is 1.

In another embodiment of the present invention there is provided a compound of formula (I) wherein R¹ is C₁₋₆alkyl, hydroxyl-C₁₋₆alkyl or benzyl; R^{1a} is hydrogen or C₁₋₆alkyl; R² is C₁₋₆alkyl; R³ is 1-naphthylmethyl, 2-naphthylmethyl, (4-phenylphenyl)methyl or indolyl-3-methyl; R^{3a} is hydrogen; R⁴ is (iic); R⁵ is hydrogen or 3-guanidopropyl; R⁶ is hydroxymethyl, (1R)-1-hydroxyethyl or 4-aminobutyl; R⁷ is C₁₋₆ alkyl or optionally substituted guanidino-C₂₋₄alkyl; R is (i); R^{8a} is hydrogen; R⁹ is (iic); R¹⁰ is C₁₋₆alkyl, amino-C₁₋₆alkyl, optionally substituted guanidino-C₂₋₄₃alkyl or (iv); R¹¹ is (iib) or (iic); R¹² is (v); R^{1a}, R^{3a}, R^{8a}, R^{15a}, R^{15b}, R^{15c}, R^{15d}, R^{15e}, R^{15f}, R^{15g}, R¹⁵ⁱ, R^{15j}, R^{15k}, R^{16a}, R^{16b}, R^{16c} and R^{16d} are hydrogen; R^{15h} is methyl.

In a subembodiment R³ is 1H-indol-3-ylmethyl; R⁴ and R⁹ is (1H-imidazol-4-yl) methyl; R⁵ is hydrogen; R⁶ is hydroxymethyl, R⁷ and R¹⁰ are 3-guanidinyl-propyl; R¹¹ is 3-methylimidazol-4-yl)methyl; R¹² is N-(2-amino-2-oxo-ethyl)-carboxamide and the remaining substituents not further limited are as described herein above.

In a subembodiment R³ is 1-naphthylmethyl; R⁴ and R⁹ is (1H-imidazol-4-yl) methyl; R⁵ is hydrogen; R⁶ is hydroxymethyl, R⁷ and R¹⁰ are 3-guanidinyl-propyl; R¹¹ is 3-methylimidazol-4-yl)methyl; R¹² is N-(2-amino-2-oxo-ethyl)-carboxamide and the remaining substituents not further limited are as described herein above.

In some embodiments there is a compound of formula (II) wherein the compound is selected from Table 2

TABLE 2

| # | Structure | Compound Name | m/e |
|---|---|---|---|
| 50 | 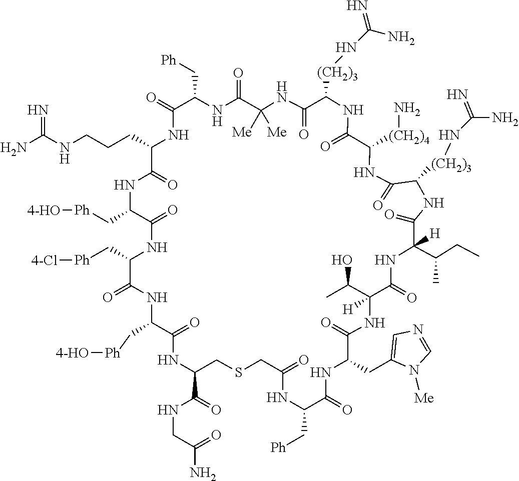 | rac- (3R,6S,9S,12S,15S,18S,21S,24S, 27S,30S,33S,36S,42S)-27-(4- aminobutyl)-N-(2-amino-2-oxo- ethyl)-6,18,42-tribenzyl-15,21,24- tris(3-guanidinopropyl)-12-[(4- hydroxyphenyl)methyl]-30-(1H- imidazol-5-ylmethyl)-9-(1H-indol- 3-ylmethyl)- 5,8,11,14,17,20,23,26,29,32,35, 38,41,44-tetradecaoxo-36-[rac- (1R)-1-hydroxyethyl]-33-[rac-(1S)- 1-methylpropyl]-1-thia- 4,7,10,13,16,19,22,25,28,31,34,37, 40,43- tetradecazacyclopentatetracontane- 3-carboxamide | 2013.01 |
| 51 | 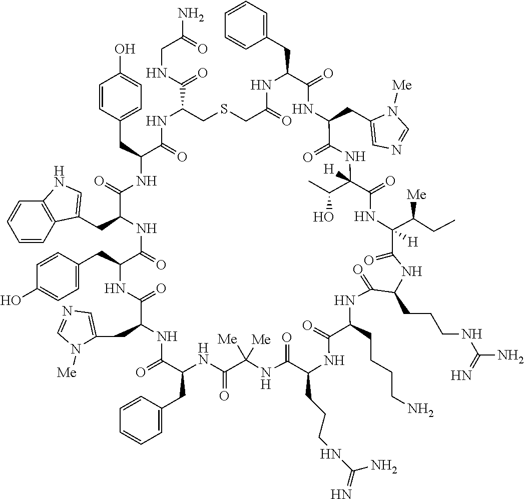 [SMILES: CC[C@H](C)[C@@H]1NC(=O)[C@H]([C@@H](C)O)NC(=O)[C@H](Cc2cncn2C)NC(=O)[C@H](Cc2ccccc2)NC(=O)CSC[C@@H](C(=O)NCC(N)=O)NC(=O)[C@H](Cc2ccc(O)cc2)NC(=O)[C@H](Cc2c[nH]c3ccccc23)NC(=O)[C@H](Cc2ccc(O)cc2)NC(=O)[C@H](Cc2cncn2C)NC(=O)[C@H](Cc2ccccc2)NC(=O)C(C)(C)NC(=O)[C@H](CCCNC(=N)N)NC(=O)[C@H](CCCCN)NC(=O)[C@H](CCCNC(=N)N)NC1=O] | (3R,6S,9S,12S,15S,18S,24S,27S, 30S,33S,36S,39S,42S)-27-(4- aminobutyl)-N-(2-amino-2-oxo- ethyl)-18,42-dibenzyl-24,30-bis(3- guanidinopropyl)-36-[(1R)-1- hydroxyethyl]-6,12-bis[(4- hydroxyphenyl)methyl]-9-(1H- indol-3-ylmethyl)-21,21-dimethyl- 15,39-bis[(3-methylimidazol-4- yl)methyl]-33-[(1S)-1- methylpropyl]- 5,8,11,14,17,20,23,26,29,32,35,38, 41,44-tetradecaoxo-1-thia- 4,7,10,13,16,19,22,25,28,31,34,37, 40,43- tetradecazacyclopentatetracontane- 3-carboxamide | 2066.02 |
| 52 | 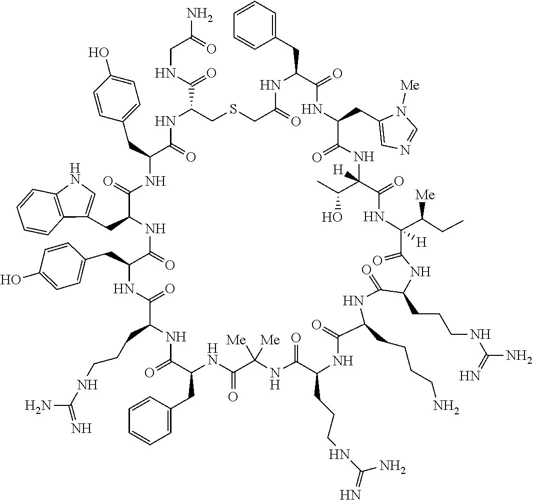 [SMILES: CC[C@H](C)[C@@H]1NC(=O)[C@H]([C@@H](C)O)NC(=O)[C@H](Cc2cncn2C)NC(=O)[C@H](Cc2ccccc2)NC(=O)CSC[C@@H](C(=O)NCC(N)=O)NC(=O)[C@H](Cc2ccc(O)cc2)NC(=O)[C@H](Cc2c[nH]c3ccccc23)NC(=O)[C@H](Cc2ccc(O)cc2)NC(=O)[C@H](CCCNC(=N)N)NC(=O)[C@H](Cc2ccccc2)NC(=O)C(C)(C)NC(=O)[C@H](CCCNC(=N)N)NC(=O)[C@H](CCCCN)NC(=O)[C@H](CCCNC(=N)N)NC1=O] | (3R,6S,9S,12S,15S,18S,24S,27S, 30S,33S,36S,39S,42S)-27-(4- aminobutyl)-N-(2-amino-2-oxo- ethyl)-18,42-dibenzyl-15,24,30- tris(3-guanidinopropyl)-36-[(1R)- 1-hydroxyethyl]-6,12-bis[(4- hydroxyphenyl)methyl]-9-(1H- indol-3-ylmethyl)-21,21-dimethyl- 39-[(3-methylimidazol-4- yl)methyl]-33-[(1S)-1- methylpropyl]- 5,8,11,14,17,20,23,26,29,32,35,38, 41,44-tetradecaoxo-1-thia- 4,7,10,13,16,19,22,25,28,31,34,37, 40,43- tetradecazacyclopentatetracontane- 3-carboxamide | 2071.05 |
| 53 | 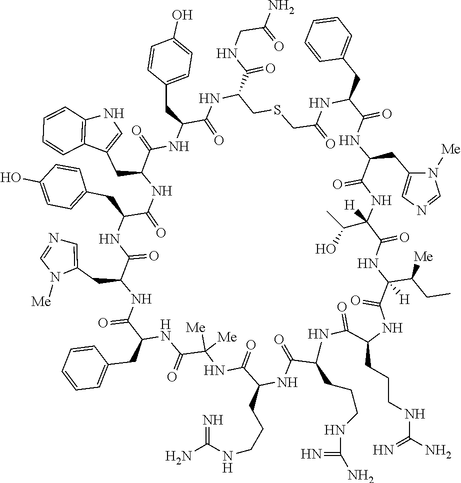 [SMILES: CC[C@H](C)[C@@H]1NC(=O)[C@H]([C@@H](C)O)NC(=O)[C@H](Cc2cncn2C)NC(=O)[C@H](Cc2ccccc2)NC(=O)CSC[C@@H](C(=O)NCC(N)=O)NC(=O)[C@H](Cc2ccc(O)cc2)NC(=O)[C@H](Cc2c[nH]c3ccccc23)NC(=O)[C@H](Cc2ccc(O)cc2)NC(=O)[C@H](Cc2cncn2C)NC(=O)[C@H](Cc2ccccc2)NC(=O)C(C)(C)NC(=O)[C@H](CCCNC(=N)N)NC(=O)[C@H](CCCNC(=N)N)NC(=O)[C@H](CCCNC(=N)N)NC1=O] | (3R,6S,9S,12S,15S,18S,24S,27S, 30S,33S,36S,39S,42S)-N-(2-amino- 2-oxo-ethyl)-18,42-dibenzyl- 24,27,30-tris(3-guanidinopropyl)- 36-[(1R)-1-hydroxyethyl]-6,12- bis[(4-hydroxyphenyl)methyl]-9- (1H-indol-3-ylmethyl)-21,21- dimethyl-15,39-bis[(3- methylimidazol-4-yl)methyl]-33- [(1S)-1-methylpropyl]- 5,8,11,14,17,20,23,26,29,32,35,38, 41,44-tetradecaoxo-1-thia- 4,7,10,13,16,19,22,25,28,31,34,37, 40,43- tetradecazacyclopentatetracontane- 3-carboxamide | 2094.03 |
| 54 | 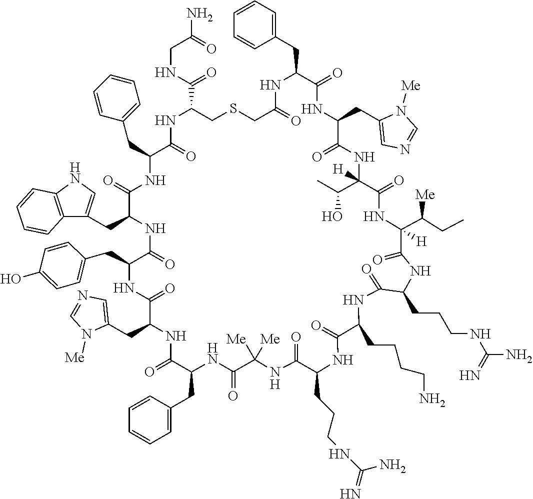 [SMILES: CC[C@H](C)[C@@H]1NC(=O)[C@H]([C@@H](C)O)NC(=O)[C@H](Cc2cncn2C)NC(=O)[C@H](Cc2ccccc2)NC(=O)CSC[C@@H](C(=O)NCC(N)=O)NC(=O)[C@H](Cc2ccccc2)NC(=O)[C@H](Cc2c[nH]c3ccccc23)NC(=O)[C@H](Cc2ccc(O)cc2)NC(=O)[C@H](Cc2cncn2C)NC(=O)[C@H](Cc2ccccc2)NC(=O)C(C)(C)NC(=O)[C@H](CCCNC(=N)N)NC(=O)[C@H](CCCCN)NC(=O)[C@H](CCCNC(=N)N)NC1=O] | (3R,6S,9S,12S,15S,18S,24S,27S, 30S,33S,36S,39S,42S)-27-(4- aminobutyl)-N-(2-amino-2-oxo- ethyl)-6,18,42-tribenzyl-24,30- bis(3-guanidinopropyl)-36-[(1R)-1- hydroxyethyl]-12-[(4- hydroxyphenyl)methyl]-9-(1H- indol-3-ylmethyl)-21,21-dimethyl- 15,39-bis[(3-methylimidazol-4- yl)methyl]-33-[(1S)-1- methylpropyl]- 5,8,11,14,17,20,23,26,29,32,35,38, 41,44-tetradecaoxo-1-thia- 4,7,10,13,16,19,22,25,28,31,34,37, 40,43- tetradecazacyclopentatetracontane- 3-carboxamide; 2,2,2- trifluoroacetic acid | 2050.03 |
| 55 | 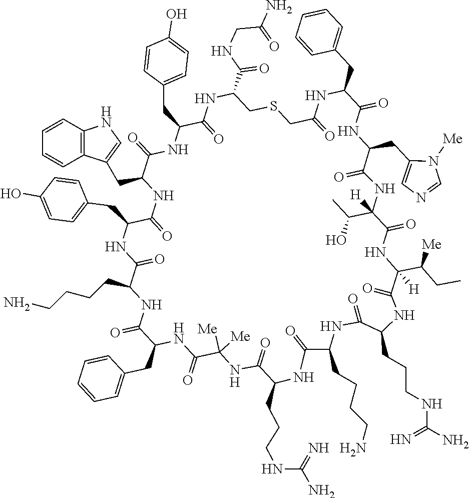 [SMILES: CC[C@H](C)[C@@H]1NC(=O)[C@H]([C@@H](C)O)NC(=O)[C@H](Cc2cncn2C)NC(=O)[C@H](Cc2ccccc2)NC(=O)CSC[C@@H](C(=O)NCC(N)=O)NC(=O)[C@H](Cc2ccc(O)cc2)NC(=O)[C@H](Cc2c[nH]c3ccccc23)NC(=O)[C@H](Cc2ccc(O)cc2)NC(=O)[C@H](CCCCN)NC(=O)[C@H](Cc2ccccc2)NC(=O)C(C)(C)NC(=O)[C@H](CCCNC(=N)N)NC(=O)[C@H](CCCCN)NC(=O)[C@H](CCCNC(=N)N)NC1=O] | (3R,6S,9S,12S,15S,18S,24S,27S, 30S,33S,36S,39S,42S)-15,27-bis(4- aminobutyl)-N-(2-amino-2-oxo- ethyl)-18,42-dibenzyl-24,30-bis(3- guanidinopropyl)-36-[(1R)-1- hydroxyethyl]-6,12-bis[(4- hydroxyphenyl)methyl]-9-(1H- indol-3-ylmethyl)-21,21-dimethyl- 39-[(3-methylimidazol-4- yl)methyl]-33-[(1S)-1- methylpropyl]- 5,8,11,14,17,20,23,26,29,32,35,38, 41,44-tetradecaoxo-1-thia- 4,7,10,13,16,19,22,25,28,31,34,37, 40,43- tetradecazacyclopentatetracontane- 3-carboxamide | 2046.04 |
| 56 | 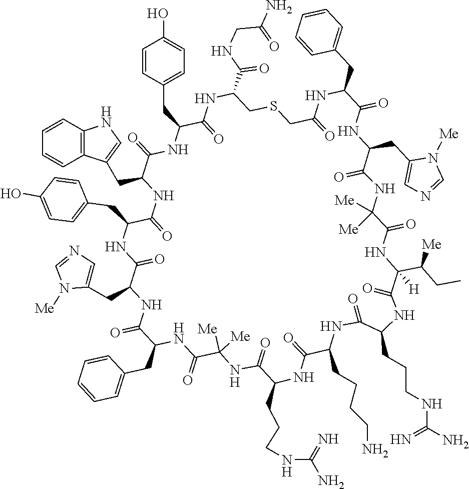 [SMILES: CC[C@H](C)[C@@H]1NC(=O)C(C)(C)NC(=O)[C@H](Cc2cncn2C)NC(=O)[C@H](Cc2ccccc2)NC(=O)CSC[C@@H](C(=O)NCC(N)=O)NC(=O)[C@H](Cc2ccc(O)cc2)NC(=O)[C@H](Cc2c[nH]c3ccccc23)NC(=O)[C@H](Cc2ccc(O)cc2)NC(=O)[C@H](Cc2cncn2C)NC(=O)[C@H](Cc2ccccc2)NC(=O)C(C)(C)NC(=O)[C@H](CCCNC(=N)N)NC(=O)[C@H](CCCCN)NC(=O)[C@H](CCCNC(=N)N)NC1=O] | (3R,6S,9S,12S,15S,18S,24S,27S, 30S,33S,39S,42S)-27-(4-aminobutyl)- N-(2-amino-2-oxo-ethyl)-18,42- dibenzyl-24,30-bis(3- guanidinopropyl)-6,12-bis[(4- hydroxyphenyl)methyl]-9-(1H- indol-3-ylmethyl)-21,21,36,36- tetramethyl-15,39-bis[(3- methylimidazol-4-yl)methyl]-33- [(1S)-1-methylpropyl]- 5,8,11,14,17,20,23,26,29,32,35,38, 41,44-tetradecaoxo-1-thia- 4,7,10,13,16,19,22,25,28,31,34,37, 40,43- tetradecazacyclopentatetracontane- 3-carboxamide | 2050.03 |
| 57 | 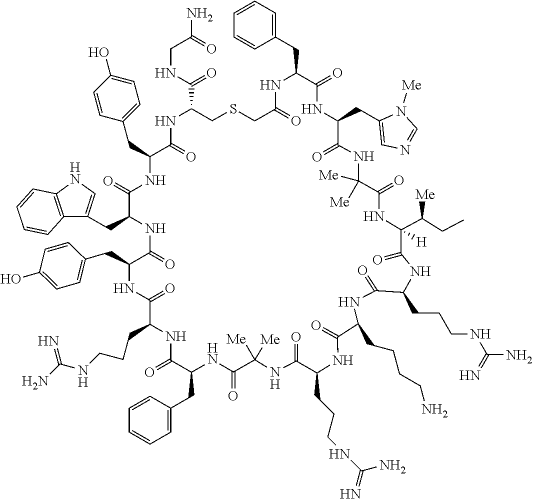 [SMILES: CC[C@H](C)[C@@H]1NC(=O)C(C)(C)NC(=O)[C@H](Cc2cncn2C)NC(=O)[C@H](Cc2ccccc2)NC(=O)CSC[C@@H](C(=O)NCC(N)=O)NC(=O)[C@H](Cc2ccc(O)cc2)NC(=O)[C@H](Cc2c[nH]c3ccccc23)NC(=O)[C@H](Cc2ccc(O)cc2)NC(=O)[C@H](CCCNC(=N)N)NC(=O)[C@H](Cc2ccccc2)NC(=O)C(C)(C)NC(=O)[C@H](CCCNC(=N)N)NC(=O)[C@H](CCCCN)NC(=O)[C@H](CCCNC(=N)N)NC1=O] | (3R,6S,9S,12S,15S,18S,24S,27S, 30S,33S,39S,42S)-27- (4-aminobutyl)- N-(2-amino-2-oxo-ethyl)-18,42- dibenzyl-15,24,30-tris(3- guanidinopropyl)-6,12-bis[(4- hydroxyphenyl)methyl]-9-(1H- indol-3-ylmethyl)-21,21,36,36- tetramethyl-39-[(3- methylimidazol-4-yl)methyl]-33- [(1S)-1-methylpropyl]- 5,8,11,14,17,20,23,26,29,32,35,38, 41,44-tetradecaoxo-1-thia- 4,7,10,13,16,19,22,25,28,31,34,37, 40,43- tetradecazacyclopentatetracontane- 3-carboxamide | 2055.05 |
| 58 | 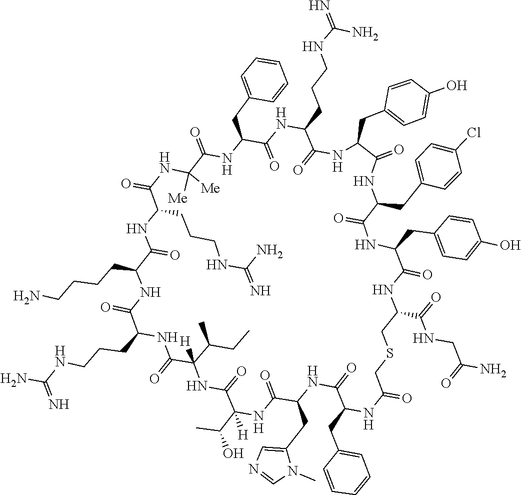 [SMILES: CC[C@H](C)[C@@H]1NC(=O)[C@H]([C@@H](C)O)NC(=O)[C@H](Cc2cncn2C)NC(=O)[C@H](Cc2ccccc2)NC(=O)CSC[C@@H](C(=O)NCC(N)=O)NC(=O)[C@H](Cc2ccc(O)cc2)NC(=O)[C@H](Cc2ccc(Cl)cc2)NC(=O)[C@H](Cc2ccc(O)cc2)NC(=O)[C@H](CCCNC(=N)N)NC(=O)[C@H](Cc2ccccc2)NC(=O)C(C)(C)NC(=O)[C@H](CCCNC(=N)N)NC(=O)[C@H](CCCCN)NC(=O)[C@H](CCCNC(=N)N)NC1=O] | (3R,6S,9S,12S,15S,18S,24S,27S, 30S,33S,36S,39S,42S)-27-(4- aminobutyl)-N-(2-amino-2-oxo- ethyl)-18,42-dibenzyl-9-[(4- chlorophenyl)methyl]-15,24,30- tris(3-guanidinopropyl)-36-[(1R)- 1-hydroxyethyl]-6,12-bis[(4- hydroxyphenyl)methyl]-21,21- dimethyl-39-[(3-methylimidazol- 4-yl)methyl]-33-[(1S)-1- methylpropyl]- 5,8,11,14,17,20,23,26,29,32,35,38, 41,44-tetradecaoxo-1-thia- 4,7,10,13,16,19,22,25,28,31,34,37, 40,43- tetradecazacyclopentatetracontane- 3-carboxamide | 2066.00 |
| 59 | 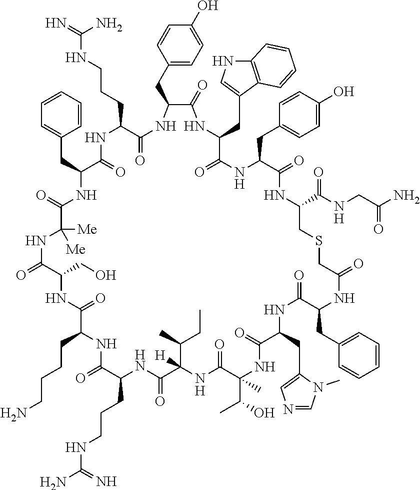 [SMILES: CC[C@H](C)[C@@H]1NC(=O)[C@](C)([C@@H](C)O)NC(=O)[C@H](Cc2cncn2C)NC(=O)[C@H](Cc2ccccc2)NC(=O)CSC[C@@H](C(=O)NCC(N)=O)NC(=O)[C@H](Cc2ccc(O)cc2)NC(=O)[C@H](Cc2c[nH]c3ccccc23)NC(=O)[C@H](Cc2ccc(O)cc2)NC(=O)[C@H](CCCNC(=N)N)NC(=O)[C@H](Cc2ccccc2)NC(=O)C(C)(C)NC(=O)[C@H](CO)NC(=O)[C@H](CCCCN)NC(=O)[C@H](CCCNC(=N)N)NC1=O] | rac- (3R,6S,9S,12S,15S,18S,24S,27S, 30S,33S,36S,39S,42S)-27-(4- aminobutyl)-N-(2-amino-2-oxo- ethyl)-18,42-dibenzyl-15,30-bis(3- guanidinopropyl)-24- (hydroxymethyl)-6,12-bis[(4- hydroxyphenyl)methyl]-9-(1H- indol-3-ylmethyl)-21,21-dimethyl- 39-[(3-methylimidazol-4- yl)methyl]- 5,8,11,14,17,20,23,26,29,32,35,38, 41,44-tetradecaoxo-36-[rac-(1R)- 1-hydroxyethyl]-33-[rac-(1S)-1- methylpropyl]-1-thia- 4,7,10,13,16,19,22,25,28,31,34,37, 40,43- tetradecazacyclopentatetracontane- 3-carboxamide | 2001.98 |
| 60 | 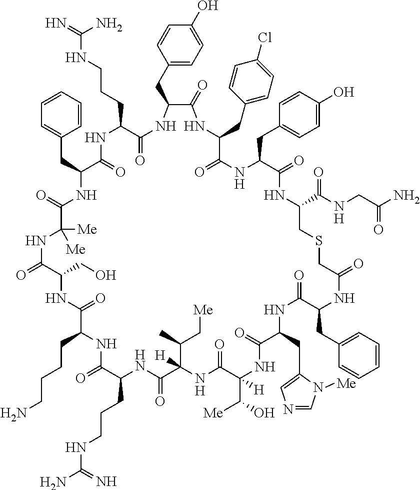 [SMILES: CC[C@H](C)[C@@H]1NC(=O)[C@H]([C@@H](C)O)NC(=O)[C@H](Cc2cncn2C)NC(=O)[C@H](Cc2ccccc2)NC(=O)CSC[C@@H](C(=O)NCC(N)=O)NC(=O)[C@H](Cc2ccc(O)cc2)NC(=O)[C@H](Cc2ccc(Cl)cc2)NC(=O)[C@H](Cc2ccc(O)cc2)NC(=O)[C@H](CCCNC(=N)N)NC(=O)[C@H](Cc2ccccc2)NC(=O)C(C)(C)NC(=O)[C@H](CO)NC(=O)[C@H](CCCCN)NC(=O)[C@H](CCCNC(=N)N)NC1=O] | (3R,6S,9S,12S,15S,18S,24S,27S, 30S,33S,36S,39S,42S)-27-(4- aminobutyl)-N-(2-amino-2-oxo- ethyl)-18,42-dibenzyl-9-[(4- chlorophenyl)methyl]-15,30-bis(3- guanidinopropyl)-24- (hydroxymethyl)-6,12-bis[(4- hydroxyphenyl)methyl]-21,21- dimethyl-39-[(3-methylimidazol- 4-yl)methyl]- 5,8,11,14,17,20,23,26,29,32,35,38, 41,44-tetradecaoxo-36-[rac-(1R)- 1-hydroxyethyl]-33-[rac-(1S)-1- methylpropyl]-1-thia- 4,7,10,13,16,19,22,25,28,31,34,37, 40,43- tetradecazacyclopentatetracontane- 3-carboxamide | 1996.93 |
| 61 | 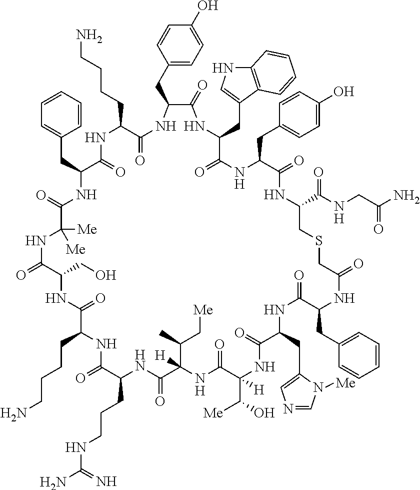 [SMILES: CC[C@H](C)[C@@H]1NC(=O)[C@H]([C@@H](C)O)NC(=O)[C@H](Cc2cncn2C)NC(=O)[C@H](Cc2ccccc2)NC(=O)CSC[C@@H](C(=O)NCC(N)=O)NC(=O)[C@H](Cc2ccc(O)cc2)NC(=O)[C@H](Cc2c[nH]c3ccccc23)NC(=O)[C@H](Cc2ccc(O)cc2)NC(=O)[C@H](CCCCN)NC(=O)[C@H](Cc2ccccc2)NC(=O)C(C)(C)NC(=O)[C@H](CO)NC(=O)[C@H](CCCCN)NC(=O)[C@H](CCCNC(=N)N)NC1=O] | rac- (3R,6S,9S,12S,15S,18S,24S,27S, 30S,33S,36S,39S,42S)-15,27-bis(4- aminobutyl)-N-(2-amino-2-oxo- ethyl)-18,42-dibenzyl-30-(3- guanidinopropyl)-24- (hydroxymethyl)-6,12-bis[(4- hydroxyphenyl)methyl]-9-(1H- indol-3-ylmethyl)-21,21-dimethyl- 39-[(3-methylimidazol-4- yl)methyl]- 5,8,11,14,17,20,23,26,29,32,35,38, 41,44-tetradecaoxo-36-[rac-(1R)- 1-hydroxyethyl]-33-[rac-(1S)-1- methylpropyl]-1-thia- 4,7,10,13,16,19,22,25,28,31,34,37, 40,43- tetradecazacyclopentatetracontane- 3-carboxamide | 1973.98 |

In some embodiments of the invention, a cyclic tetradecapeptide of Formula (IIa), or a pharmaceutically acceptable salt, solvate, or stereoisomer thereof:

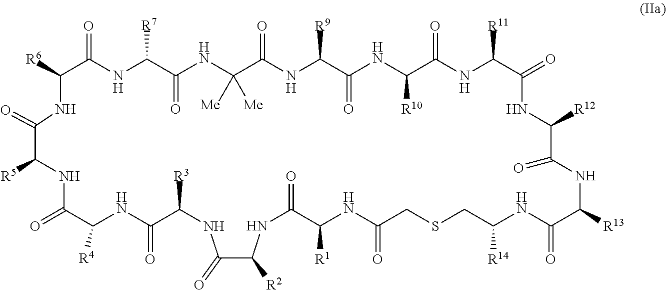

In one embodiment of a compound of Formula (IIa), R¹⁴ is N-(2-amino-2-oxo-ethyl)carboxamide (ν, r=1, R^{e}=H); R⁴ is (1S)-1-methylpropyl, R² is 1-methyl-1H-imidazol-5-yl)methyl and R⁹ is benzyl optionally substituted with halo or hydroxyl. In one embodiment of a compound of Formula (II) R⁹ is unsubstituted benzyl.

In one embodiment of the present invention there is a compound of formula (IIb) wherein:

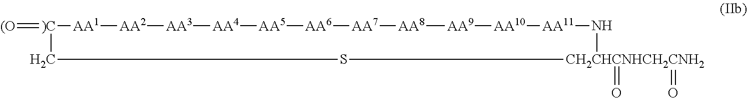

AA¹ is an aromatic amino acid wherein the aromatic amino acid is phenylalanine optionally substituted by 1 to 3 C₁₋₆ alkyl, halogens, C₁₋₆-haloalkyl, hydroxyl, C₁₋₆-alkoxy, C₁₋₆-haloalkoxy, C₁₋₆-thioalkyl, C₁₋₆-alkylsulfonyl or cyano;
AA² is a heteroaryl amino acid wherein said heteroaryl amino acid is a compound of formula H₂NCHR^{a}CO₂H wherein R^{a} is 1H-imidazole-5-ylmethyl, 3-methylimidazol-4-yl-methyl, preferably R^{a} is 3-methylimidazol-4-yl-methyl or AA² is glycine;
AA³ is an aliphatic amino acid of formula H₂NCHR^{a}CO₂H wherein R^{a} is C₁₋₆ alkyl or C₁₋₅-hydroxyalkyl or an aliphatic amino acid of formula H₂NCR^{a}R^{b}CO₂H wherein R^{a} and R^{b} are independently C₁₋₆ alkyl; preferably α-aminoisobutyric acid (Aib);
AA⁴ is an aliphatic amino acid of formula H₂NCHR^{a}CO₂H wherein R^{a} is C₁₋₆ alkyl, preferably, AA⁴ is isoleucine;
AA⁵ is a basic amino acid of formula H₂NCHR^{a} CO₂H wherein R^{a} is H-imidazole-5-ylmethyl or guanidino-C₁₋₄-alkyl, preferably arginine;
AA⁶ is a basic amino acid of formula H₂NCHR^{a} CO₂H wherein R^{a} is C₁₋₆-aminoalkyl or guanidino-C₁₋₄-alkyl, preferably, aminobutyl;
AA⁷ is a basic amino acid of formula H₂NCHR^{a} CO₂H wherein R^{a} is C₁₋₆-hydroxyalkyl or guanidino-C₁₋₄-alkyl, preferably, aminobutyl;
AA⁸ is an aliphatic amino acid of formula H₂NCR^{a}R^{b}CO₂H wherein R^{a} is C₁₋₆ alkyl and R^{b} hydrogen or C₁₋₆ alkyl; preferably α-aminoisobutyric acid (Aib);
AA⁹ is an aromatic amino acid wherein the aromatic amino acid is phenylalanine wherein the phenyl ring of either is optionally substituted by 1 to 3 C₁₋₆ alkyl, halogens, C₁₋₆-haloalkyl, hydroxyl, C₁₋₆-alkoxy, C₁₋₆-haloalkoxy, C₁₋₆-thioalkyl; preferably phenylalanine;
AA¹⁰ is a basic amino acid of formula H₂NCHR^{a} CO₂H wherein R^{a} is C₁₋₆-aminoalkyl, guanidino-C₁₋₄-alkyl, 3-methylimidazol-4-yl-methyl;
AA¹¹ is an aromatic amino acid wherein the aromatic amino acid is phenylalanine wherein the phenyl ring of either is optionally substituted by 1 to 3 C₁₋₆ alkyl, halogens, C₁₋₆-haloalkyl, hydroxyl, C₁₋₆-alkoxy, C₁₋₆-haloalkoxy, C₁₋₆-thioalkyl; preferably phenylalanine;
AA¹² is a heteroaryl amino acid of formula H₂NCHR^{a} CO₂H wherein R^{a} is 3-indolemethyl or aromatic amino acid wherein R^{a} is phenyl ring optionally substituted by 1 to 3 C₁₋₆ alkyl, halogens, C₁₋₆ haloalkyl, hydroxyl, C₁₋₆-alkoxy, C₁₋₆-haloalkoxy, C₁₋₆-thioalkyl; preferably tryptophan;
AA¹³ is phenylalanine wherein the phenyl ring is optionally substituted by one to three C₁₋₆ alkyl, halogens, C₁s-haloalkyl, C₁₋₆-alkoxy, C₁₋₆-haloalkoxy, C₁₋₆-thioalkyl, C₁₋₆-alkylsulfonyl or cyano.

In another embodiment there is afforded a compound of formula (IIb) wherein: AA¹ is phenylalanine; AA² is N^{α}-methylhistidine; AA³ is serine or alanine; AA⁴ is isoleucine; AA⁵ is arginine or histidine; AA⁶ is lysine or arginine; AA⁷ is arginine or serine; AA⁸ is α-aminoisobutyric acid; AA⁹ is phenylalanine; AA¹⁰ is arginine, N^{α}-methylhistidine or H₂NCHR^{a}CO₂H wherein R^{a} is amino methyl, aminoethyl or aminobutyl; AA¹¹ is 4-hydroxy-phenylalanine; AA¹² is tryptophan; AA¹³ is phenylalanine optionally substituted with hydroxyl or halo.

### Dosage & Administration

The present invention provides pharmaceutical compositions or medicaments containing the compounds of the invention and at least one therapeutically inert carrier, diluent or excipient, as well as methods of using the compounds of the invention to prepare such compositions and medicaments. In one example, compounds of Formula I with the desired degree of purity may be formulated by mixing with physiologically acceptable carriers, i.e., carriers that are non-toxic to recipients at the dosages and concentrations employed into a dosage form at ambient temperature and at the appropriate pH. The pH of the formulation depends mainly on the particular use and the concentration of compound, but typically ranges anywhere from about 3 to about 8. In one example, a compound of Formula I is formulated in an acetate buffer, at pH 5. In another embodiment, the compounds of Formula I are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the severity of the disorder, the particular patient being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to provides anti-bacterial activity. Typically such amount may be below the amount that is toxic to normal cells, or the patient as a whole.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Sustained-release preparations may be prepared. Suitable examples of sustained-release preparations include semipermeable matrices of solid hydrophobic polymers containing a compound of Formula I, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides, copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A dose to treat human patients may range from about 0.1 mg to about 1000 mg of a compound of formula I or II. A typical dose may be about 1 mg to about 300 mg of the compound. A dose may be administered once a day (QD), twice per day (BID), or more frequently, depending on the pharmacokinetic and pharmacodynamic properties, including absorption, distribution, metabolism, and excretion of the particular compound. In addition, toxicity factors may influence the dosage and administration regimen.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal, epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, H. C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, R. C., Handbook of Pharmaceutical Excipients, Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

In another aspect are methods of treating a mammal in need of such treatment comprising administering to the mammal an antibacterial effective amount of any of the aforementioned compounds at a frequency and for a duration sufficient to provide a beneficial effect to the mammal. In one embodiment, the causative bacteria species of the bacteria infection is an infection involving a Gram-negative bacteria. In such embodiments, the gram-negative bacteria may be, for example, Escheria coli, Klebsiella pneumoniae, Pseudomonas aeruginosa, Acinetobacter baumanii, Neisseria gonorrhoeae, Neisseria meningitidis, Chlamvdia trachomatis, Moraxella catarrhalis, Haemophilus influenzae, Proteus mirabilis, Enterobacter cloacae, Serratia marcescens, Helicobacter pylori, Salmonella enteritidis, Salmonella tvphi, Legionella pneumophila, Haemophilus influenzae, Vibrio cholerae, Pseudomonas stutzeri, Ralstonia solanacearum, or Xylella fastidiosa.

In another aspect the bacterial infection is an infection involving a Gram-negative bacteria wherein the bacteria is E. coli.

Injectable preparations, for example, sterile injectable aqueous or oleaginous suspensions are optionally formulated according to the known art using suitable dispersing or wetting agents and suspending agents. The sterile injectable preparation is optionally a sterile injectable solution, suspension or emulsion in a nontoxic parenterally acceptable diluent or solvent, for example, as a solution in 1,3-butanediol. Among the acceptable vehicles and solvents that are optionally employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

According to the methods of treatment described herein, bacterial infections are treated or prevented in a patient such as a human or lower mammal by administering to the patient a therapeutically effective amount of a compound described herein, in such amounts and for such time as is necessary to achieve the desired result. By a “therapeutically effective amount” of a compound described herein is meant a sufficient amount of the compound to treat bacterial infections, at a reasonable benefit/risk ratio applicable to any medical treatment. It will be understood, however, that the total daily usage of the compounds and compositions described herein will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors known in the medical arts.

The total daily dose of the compounds described herein compound described herein (i.e., a compound disclosed herein) administered to a human or other mammal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens described herein comprise administration to a patient in need of such treatment from about 10 mg to about 2000 mg of the compound(s) described herein per day in single or multiple doses.

### Experimental Procedures

All starting materials, building blocks, reagents, acids, bases, solid phase resins, and solvents used in in the following examples were commercially available products were typically used without any further purification. Standard techniques were used if purification was required. Unless otherwise specified, commercially available protected amino acids were used without additional purification.

The structure cyclic peptides was confirmed by ESI-MS (+) in the mass spectrum analysis. ESI-MS (+) indicates an electrospray ionization mass spectrum analysis method performed in the positive ion mode Cyclic peptides having a molecular weight greater than about 1000 were frequently detected as divalent ions or trivalent ions.

Peptide synthesis carried by standard solid-phase synthesis methodology using Sieber Amide resin or Fmoc-NH-SAL-PEG resin and Fmoc-protected amino acids. Coupling steps typically carried out with 5.2 equivalents of Fmoc-amino acid, 5 equivalents of HATU, and 10 equivalents of DIEA as coupling reagents with automated Liberty Blue automated peptide synthesizer (CEM Inc.) or with 3.2 equivalents of Fmoc-amino acid, 3 equivalents of HATU, and 6 equivalents of DIEA as coupling reagents with Syro I automated Parallel Peptide Synthesizer (Biotage) for peptide elongation. Deprotection was carried either with 20% piperidine in DMF or 5% piperazine in DMF with 0.1 M HOBt.

Cyclic peptides were purified by reverse-phase high-performance liquid chromatography (HPLC) using Waters AutoPurification System or Shimadzu prep-HPLC system. The peptide thus obtained was identified by the mass spectrum obtained in the ESI-positive scan mode and the mass spectrum containing polyvalent ions calculated from the molecular formula of the target object were in agreement within the error range of the mass spectrometer used.

Analysis LCMS conditions: Column: Kinetex EVO C18 100 Å 2.6 μm, 2.1 mm ID×150 mm column eluted with 0.025% TFA in water (Mobil Phase A) and 0.025% TFA in MeCN (Mobile phases B) with 5-45% gradient of B over 20 minutes and a 0.25 mL/min flow rate. Eluents were monitored with UV 225 nm detector.

### REPRESENTATIVE EXAMPLES

### Example 1: Compound 16

Fmoc amino acids used in the synthesis included Fmoc-Gly-OH; Fmoc-Cys(Trt)-OH; Fmoc-H3Me-OH; Fmoc-Arg(Pbf)-OH; Fmoc-His(Boc)-OH; Fmoc-MeTyr(tBu)-OH; Fmoc-Ser(Trt)-OH; Fmoc-Nal1-OH; Fmoc-Abu-OH; Fmoc-Phe-OH.

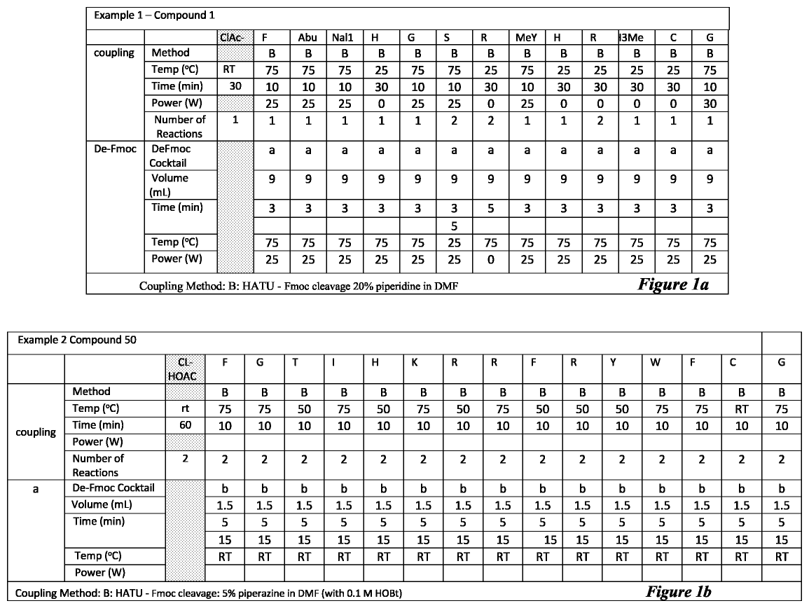

The automated synthesizer program is shown in FIG. 1a.

The synthesis was carried out on Sieber Amide resin using 5.2 equivalents of Fmoc-amino acid, 5 equivalents of HATU, and 10 equivalents of DIEA as coupling reagents; 20% piperidine in DMF for Fmoc deprotection on a and Liberty Blue for peptide synthesizer. Double couplings were used at positions 6, 7, and 10 from N-terminal amino acid.

After deprotection of the N-terminal amino acid the resin was washed with DMF, then 5 equiv of 2-chloroacetic acid, 5 equivalents of HATU, and 10 equivalents of DIEA in DMF were added and the resulting mixture was agitated for 30 minutes. The resin was successively washed with DMF and DCM and then dried under high vacuum. Global deprotection was carried out by adding a mixture of TFA/water/TIS/DODT (92.5:2.5:2.5:2.5, v/v/v/v) and agitating the resulting mixture at RT for 1.5 h. The resulting solution was filtered through a disposable filter column and diluted in diisopropyl ether-hexane (1:1, v/v). The precipitated solid was centrifuged and the supernatant solution was decanted. The remaining solid was thrice washed with Et₂O and dried under high vacuum. The crude peptide was dissolved in DMSO to give a final concentration of 5 mM and 10 equivalents of TEA were added. The mixture was stirred at RT for 18 h.

The crude material was purified via the reverse-phase HPLC with a XSelect C18 5 μm 19 mm×150 mm column. The product was eluted with 0.1% TFA in water (Mobile Phase A) and 0.1% TFA in MeCN (Mobile Phase B) with a program gradient of 5-17% B over 3 min, 17-22% B over 8 min, then 22-60% B over 1 min. The flow rate was 17 mL/min with a column temperature of 40° C. The retention time was 9.06 min. Fractions containing the desired product were combined and dried via lyophilization. MS (ESI+) m/z 853.93 [M+2H]²⁺

### Example 2 Compound 50

Fmoc amino acids used in the synthesis included Fmoc-Gly-OH; Fmoc-Cys(Trt)-OH; Fmoc-Phe-OH; Fmoc-Trp(Boc)-OH; Fmoc-Tyr(tBu)-OH; Fmoc-Arg(Pbf)-OH; Fmoc-Lys(Boc)-OH; Fmoc-His(Trt)-OH; Fmoc-Ile-OH; Fmoc-Thr(tBu)-OH.

The automated synthesizer program is shown in FIG. 1b.

The synthesis was carried out on Sieber Amide resin; 3.2 equivalents of Fmoc-amino acid, 3 equivalents of HATU, and 6 equivalents of DIEA as coupling reagents; 5% piperazine in DMF with 0.1 M HOBt was utilized for Fmoc deprotection on a Syro I for automated peptide synthesizer. Double couplings were used at all positions.

After deprotection of the N-terminal Fmoc-Phe-OH, the resin was washed with DMF, then 3.2 equivalents of 2-chloroacetic acid, 3 equivalents of HCTU, and 6 equivalents of DIEA in DMF were added and the resulting mixture was allowed to agitated for 20 min. The resin was successively washed with DMF and DCM and then dried under high vacuum. A TFA-water-TIS-DODT mixture (92.5:2.5:2.5:2.5, v/v/v/v) was added and the resulting mixture was agitated at RT for 2 h. The solution was filtered through a disposable filter column and diluted in Et₂O-hexane (1:1, v/v). The precipitated solid was centrifuged and the supernatant solution was decanted and the resulting solid was thrice washed with Et₂O and dried under high vacuum. The crude peptide was dissolved in DMSO-50% aqueous IPA (10:1, v/v) to give a final concentration of 5 mM and 5 equiv of TEA was added. The mixture was stirred at RT for 18 h.

The crude material was purified via the reverse-phase HPLC with a XBridge C18 5 μm 50 mm×150 mm column. The product was eluted with 0.1% TFA in water (Mobile Phase A) and 0.1% TFA in MeCN (Mobile Phase B) with a program gradient 5-26% B over 3 minutes, 26-31% B over 8 minutes, then 31-60% B over 1 min. The flow rate was 120 mL/min. The retention time was 13.83 min with a column temperature of 40° C. Fractions containing the desired product were combined and dried via lyophilization. MS (ESI+) m/z 1007.54 [M+2H]²⁺

### Example 3—Compound 1

Fmoc amino acids used in the synthesis included Fmoc-Gly-OH; Fmoc-Cys(Trt)-OH; Fmoc-His(Trt)-OH; Fmoc-Arg(Pbf)-OH; Fmoc-MePhe-OH; Fmoc-Ser(tBu)-OH; Fmoc-Trp(Boc)-OH; Fmoc-Val-OH; Fmoc-Phe-OH.

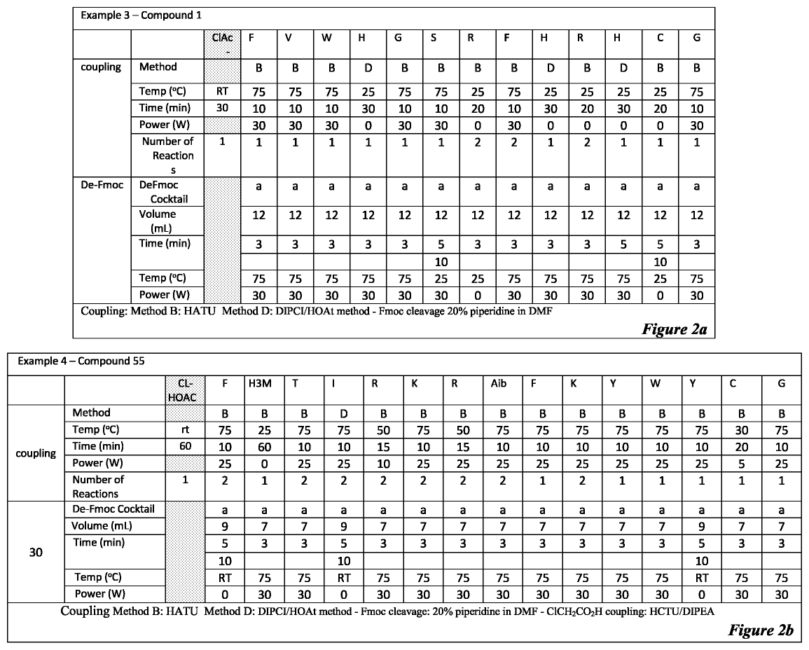

The automated synthesizer program is shown in FIG. 2a.

The crude material was purified via the reverse-phase HPLC with a Kinetex EVO C18 21.2 mm×150 mm column and eluted with 0.1% TFA in water (Mobile Phase A) and 0.1% TFA in MeCN (Mobile Phase B) with a program gradient of 5-15% B over 3 minutes, 15-20% B over 8 minutes, then 20-60% B over 1 minute. The flow rate was 21 mL/min. The retention time was 13.83 min with a column temperature of 40° C. Fractions containing the desired product were combined and lyophilized. MS (ESI+) m z 840.31 [M+2H]²⁺⁺

### Example 4—Compound 55

Fmoc amino acids used in the synthesis included Fmoc-Gly-OH; Fmoc-Cys(Trt)-OH; Fmoc-Tyr(tBu)-OH; Fmoc-Trp(Boc)-OH; Fmoc-Lys(Boc)-OH; Fmoc-Phe-OH; Fmoc-Aib-OH; Fmoc-Arg(Pbf)-OH; Fmoc-Ile-OH; Fmoc-Thr(tBu)-OH; Fmoc-H3Me-OH.

The automated synthesizer program is shown in FIG. 2ba.

The crude material was purified via the reverse-phase HPLC with a XBridge C18 5 μm 30 mm×150 mm column and eluted with 0.1% TFA in water (Mobile Phase A) and 0.1% TFA in MeCN (Mobile Phase B) with a program gradient of 5-21% B over 3 minutes, 21-26% B over 8 minutes, then 26-60% B over 1 minute. The flow rate was 45 mL/min. Fractions containing the desired product were combined and dried via lyophilization. MS (ESI+) m/z 1022.57 [M+2H]²⁺

### Example 5—Compound 61

Fmoc amino acids used in the synthesis included Fmoc-Gly-OH; Fmoc-Cys(Trt)-OH; Fmoc-Tyr(tBu)-OH; Fmoc-Trp(Boc)-OH; Fmoc-Lys(Boc)-OH; Fmoc-Phe-OH; Fmoc-Aib-OH; Fmoc-Ser(tBu)-OH; Fmoc-Arg(Pbf)-OH; Fmoc-Ile-OH; Fmoc-Thr(tBu)-OH; Fmoc-H3Me-OH.

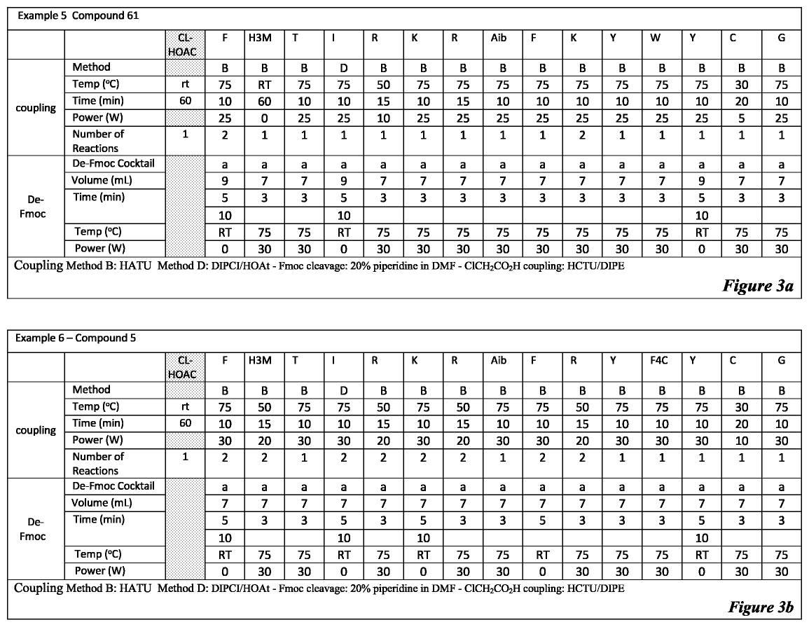

The automated synthesizer program is shown in FIG. 3a.

The crude material was purified via the reverse-phase HPLC with a XBridge C18 5 μm 19 mm×150 mm column and eluted with 0.1% TFA in water (Mobile Phase A) and 0.1% TFA in MeCN (Mobile Phase B) with a program gradient of 5-21% B over 3 minutes, 21-26% B over 8 minutes, then 26-60% B over 1 minute. The flow rate was 45 mL/min. Fractions containing the desired product were combined and lyophilized. MS (ESI+) m/z 988.07 [M+2H]²⁺

### Example 6—Compound 58

Fmoc amino acids used in the synthesis included Fmoc-Gly-OH; Fmoc-Cys(Trt)-OH; Fmoc-Tyr(tBu)-OH; Fmoc-F4C—OH; Fmoc-Lys(Boc)-OH; Fmoc-Phe-OH; Fmoc-Aib-OH; Fmoc-Arg(Pbf)-OH; Fmoc-Ile-OH; Fmoc-Thr(tBu)-OH; Fmoc-H3Me-OH.

The automated synthesizer program is shown in FIG. 3b.

The crude material was purified via the reverse-phase HPLC with a XBridge C18 5 μm 30 mm×150 mm column and eluted with 0.1% TFA in water (Mobil Phase A) and 0.1% TFA in MeCN (Mobile Phase B) with a program gradient of 5-24% B over 3 minutes, 24-29% B over 8 minutes, then 29-60% B over 1 minute. The flow rate was 45 mL/min. Fractions containing the desired product were combined and lyophilized. MS (ESI+) m/z 1034.23 [M+2H]²⁺

### Biological Assays

### Minimum Inhibitory Concentration (MIC) Determination

MICs were determined by performing two-fold serial dilutions of peptides in LB broth to a final volume of 0.1 ml in round-bottom 96-well assay plates (Corning Life Sciences No 3788). Peptides were initially resuspended in 100% DMSO to 10 mM and subsequently diluted in LB medium to the appropriate concentration. Each well was inoculated with 5×10⁵ CFU/ml of the screening stain and incubated at 37° C. without agitation for 18 hours. Plates were scored by eye, and the lowest compound concentration preventing visible growth was determined to be the MIC.

TABLE 3

| | MIC | MIC | MIC | MIC | MIC |
|---|---|---|---|---|---|
| | E. coli | E. cloacae | K. pneumoniae | E. coli | E. coli |
| compd | 25922 | ATCC222 | ATCC43816 | BW25113 | 25922 IMP |
| # | WUXI::MIC | WUXI::MIC | Wuxi::MIC | WUXI::MIC | WUXI::MIC |
| 1 | 50 | 6.3 | 50 | 100 | 0.031 |
| 2 | | | | >32 | 0.008 |
| 3 | | | | >32 | 0.064 |
| 4 | | | | >32 | 0.128 |
| 5 | | | | >32 | 0.016 |
| 6 | | | | 2 | <0.000125 |
| 7 | | | | 2 | 0.0005 |
| 8 | | | | >32 | 4 |
| 9 | | | | 8 | 9.5367E−07 |
| 10 | | | | 8 | 2.3842E−07 |
| 11 | | | | 8 | 1.9074E−06 |
| 12 | | | | 1 | 3.8137E−06 |
| 13 | | | | 4 | 0.00 |
| 14 | 25 | 1.19 | 25 | 37.5 | 0.0119 |
| 15 | 50 | 3.1 | 75 | 100 | 0.016 |
| 16 | 27.32 | 0.2633 | 8.5333 | 14.767 | 1.576725 |
| 17 | 13 | 3.1 | 37.5 | 50 | 0.002 |
| 18 | 9.65 | 0.78 | 19 | 19 | 0.00585 |
| 19 | 3.1 | 0.39 | 6.3 | 6.3 | 0.0039 |
| 20 | 13 | 1.6 | 25 | 25 | 0.0078 |
| 21 | 50 | 3.1 | 25 | 50 | 0.063 |
| 22 | 6.3 | 0.39 | 13 | 25 | 0.002 |
| 23 | 25 | | | | 0.016 |
| 24 | 13 | | | | 0.016 |
| 25 | 25 | | | | 0.031 |
| 26 | 3.1 | 0.78 | 25 | 6.3 | 0.0039 |
| 27 | 3.1 | 0.39 | 13 | 13 | 0.002 |
| 28 | 3.1 | 0.2 | 3.1 | 6.3 | 0.002 |
| 29 | 1.6 | 0.1 | 3.1 | 6.3 | 0.001 |
| 30 | 25 | 1.6 | 100 | 50 | 0.016 |
| 31 | 25 | 6.3 | 50 | 50 | 0.016 |
| 32 | 25 | | | | 0.031 |
| 33 | 13 | 1.6 | 25 | 25 | 0.0005 |
| 34 | 41.667 | 6.3 | 25 | 100 | 0.063 |
| 35 | 100 | 100 | 100 | 100 | 3.1 |
| 36 | 100 | | | | 100 |
| 37 | 100 | 100 | 100 | 100 | 25 |
| 38 | 100 | 13 | 100 | 100 | 0.063 |
| 39 | 100 | 100 | 100 | 100 | 6.3 |
| 40 | | | | | |
| 41 | >100 | | | | |
| 42 | 100 | | | | |
| 43 | >100 | | | | |
| 44 | >100 | | | | |
| 45 | >100 | | | | |
| 46 | 100 | | | | |
| 47 | 12.5 | | | | |
| 48 | >100 | | | | |
| 49 | 50 | | | | |
| 50 | 2 | >64 | 32 | 2 | 1 |
| 51 | 8 | >32 | >32 | 8 | 0.5 |
| 52 | | | | 1 | 0.125 |
| 53 | | | | 4 | 0.25 |
| 54 | | | | 8 | 0.5 |
| 55 | | | | 2 | 0.25 |
| 56 | | | | >32 | 8 |
| 57 | | | | >32 | 2 |
| 58 | | | | 2 | 0.25 |
| 59 | 8 | | | 16 | 1 |
| 60 | 16 | | | 16 | 1 |
| 61 | 16 | | | 16 | 1 |

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

The patents, published applications, and scientific literature referred to herein establish the knowledge of those skilled in the art and are hereby incorporated by reference in their entirety to the same extent as if each was specifically and individually.

We claim:

1. A compound of formula I 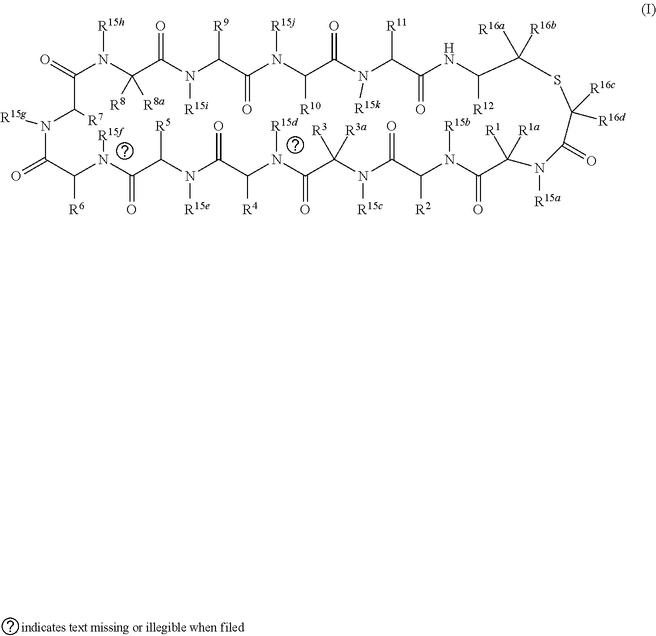
or a stereoisomer or a pharmaceutically acceptable salt thereof, wherein:
R¹ is C₁₋₆ alkyl, hydroxyl-C₁₋₆alkyl or benzyl optionally substituted with halogen or hydroxy;
R^{1a} is hydrogen;
R² is hydrogen, C₁₋₆ alkyl or optionally substituted (imidazol-4-yl)methyl;
R³ is bicyclic aryl, bicyclic heteroaryl, C₁₋₆alkyl, or hydroxyl-C₁₋₆alkyl wherein the bicyclic aryl and bicyclic heteroaryl is optionally substituted once or twice with C₁₋₆alkyl, hydroxyl or halo;
R^{3a} is hydrogen or C₁₋₆alkyl;
R⁴ is C₁₋₆alkyl or optionally substituted (imidazol-4-yl)methyl;
R⁵ is hydrogen; C₁₋₆alkyl, hydroxyl-C₁₋₆alkyl, optionally substituted (imidazol-4-yl)methyl or optionally substituted guanidino-C₂₋₄alkyl;
R⁶ is hydroxyl-C₁₋₆alkyl or amino-C₁₋₆alkyl;
R⁷ is C₁₋₆alkyl, hydroxyl-C₁₋₆alkyl or optionally substituted guanidino-C₂₋₄alkyl
R⁸ is optionally substituted benzyl;
R^{8a} is hydrogen or C₁₋₆alkyl;
R⁹ is optionally substituted benzyl or optionally substituted 1H-imidazol-5-ylmethyl;
R¹⁰ is C₁₋₆alkyl, amino-C₁₋₆alkyl, optionally substituted 1H-imidazol-5-ylmethyl, optionally substituted guanidino-C₂₋₄alkyl or optionally substituted C₁₋₂alkyl-carboxamide;
R¹¹ is optionally substituted benzyl or optionally substituted (imidazol-4-yl)methyl;
R¹² is N-(3-amino-3-oxo-propyl)carboxamide or N-(2-amino-2-oxo-ethyl)carboxamide both optionally substituted;
R^{b}, R^{c}, R^{d}, R^{e}, R^{15a}, R^{15b}, R^{15c}, R^{15d}, R^{15e}, R^{15f}, R^{15g}, R^{15h}, R¹⁵ⁱ, R^{15j}, R^{15k}, R^{16a}, R^{16b}, R^{16c} and R^{16d} are each independently hydrogen, or C₁₋₆alkyl;
R^{15e} is hydrogen, C₁₋₆alkyl, amino-C₁₋₆alkyl, or optionally substituted C₁₋₂alkyl-carboxamide;
R^{a} is hydrogen, C₁₋₆alkyl, hydroxyl or halo;
n and p are independently from 0 to 2;
q is 0 or 1; and,
r is 1 or 2.

2. The compound of claim 1 wherein R¹² is N-(2-amino-2-oxo-ethyl)carboxamide, R⁴ and R⁹ are 1H-imidazol-5-ylmethyl and R² is C₁₋₆ alkyl.

3. The compound of either of claim 1 or 2 wherein:
R¹ is C₁₋₆alkyl, hydroxyl-C₁₋₆alkyl or optionally substituted benzyl;
R³ is 1-naphthylmethyl, 2-naphthylmethyl, (4-phenylphenyl)methyl or optionally substituted 1H-indolyl-3-methyl;
R⁵ is hydrogen, or 3-guanidopropyl;
R⁶ is hydroxyl-C₁₋₆alkyl or 4-aminobutyl;
R⁷ is 3-guanidopropyl;
R¹⁰ is 3-guanidino-propyl, methyl, ethyl, aminomethyl, aminoethyl, 4-aminobutyl, 3-methylimidazol-4-yl)methyl or 3-amino-3-oxo-propyl;
R¹¹ is 1H-imidazol-5-ylmethyl or 3-methylimidazol-4-yl)methyl;
R^{b}, R^{c}, R^{e}, R^{1a}, R^{3a}, R^{8a}, R^{15b}, R^{15c}, R^{15d}, R^{15f}, R^{15g}, R¹⁵ⁱ, R^{15k}, R^{16a}, R^{16b}, R^{16c} and R^{16d} are hydrogen;
R^{15a}, R^{15e}, R^{15h} and R^{15j} are independently hydrogen or methyl;
R^{b} and R^{f} are independently hydrogen or methyl;
R^{15h} is methyl;
or, a stereoisomer or a pharmaceutically acceptable salt thereof.

4. The compound of any of claims 1 to 3 wherein:
R¹ is benzyl optionally substituted halogen or hydroxy;
R² is hydrogen, ethyl, isopropyl or (1S)-1-methylpropyl;
R³ is 1-naphthylmethyl or 1H-indole-3-methyl;
R⁴ is 1H-imidazol-5-ylmethyl;
R⁵ is hydrogen;
R⁶ is hydroxymethyl or (1R)-1-hydroxyethyl;
R⁷ is 3-guanidinopropyl;
R⁸ is benzyl or 4-hydroxybenzyl;
R⁹ is 1H-imidazol-5-ylmethyl;
R¹⁰ is 3-guanidopropyl, methyl or ethyl;
R¹¹ is 1H-imidazol-5-ylmethyl, 3-methylimidazol-4-yl)methyl;
R¹² is —C(═O)NHCH₂C(═O)NH₂;
or, a stereoisomer or a pharmaceutically acceptable salt thereof.

5. The compound of any of claims 1 to 4 wherein:
R¹ is benzyl;
R² is ethyl or isopropyl;
R³ is 1-naphthylmethyl;
R⁴ is 1H-imidazol-5-ylmethyl;
R⁵ is hydrogen;
R⁶ is hydroxylmethyl;
R⁷ is 3-guanidopropyl;
R⁵ is 4-hydroxylbenzyl or 4-chlorobenzyl;
R⁹ is 1-methyl-1H-imidazole-5-methylene;
R¹⁰ is 3-guanidopropyl;
R¹¹ is (3-methylimidazol-4-yl)methyl;
R¹² is N-(2-amino-2-oxo-ethyl)carboxamide;
R^{b} and R^{f} are independently in each occurrence hydrogen or methyl; and,
or a stereoisomer or a pharmaceutically acceptable salt thereof.

6. The compound of any one of claims 1 to 5 wherein the compound is of formula Ia; 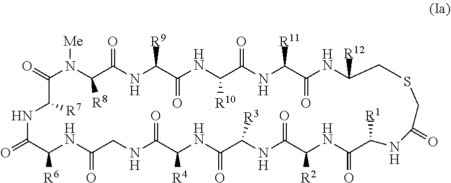
or a stereoisomer or a pharmaceutically acceptable salt thereof.

7. The compound according to any one of claims 1 to 6 wherein said compound is selected from TABLE 1.

8. A compound of Formula (II) 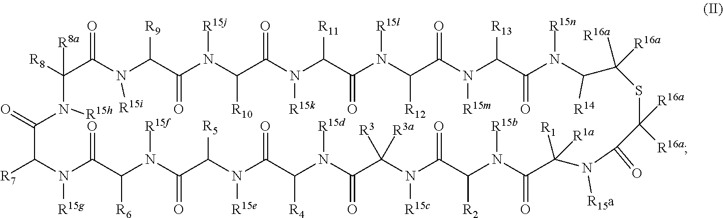
or a stereoisomer or a pharmaceutically acceptable salt thereof, wherein:
R¹ is optionally substituted benzyl;
R^{1a} is hydrogen or C₁₋₆alkyl;
R² is hydrogen or optionally substituted (imidazol-4-yl)methyl;
R³ is C₁₋₆alkyl or hydroxyl-C₁₋₆alkyl;
R^{3a} is hydrogen or C₁₋₆alkyl;
R⁴ is C₁₋₆alkyl or optionally substituted (imidazol-4-yl)methyl;
R⁵ is optionally substituted guanidino-C₂₋₄alkyl or optionally substituted (imidazol-4-yl)methyl;
R⁶ is amino-C₁₋₆alkyl or optionally substituted guanidino-C₂₋₄alkyl;
R⁷ is hydroxyl-C₁₋₆alkyl or optionally substituted guanidino-C₂₋₄alkyl;
R⁸ is methyl or 3-propylguanidine;
R^{8a} is hydrogen or methyl;
R⁹ is optionally substituted benzyl;
R¹⁰ is amino-C₁₋₆alkyl, optionally substituted (imidazol-4-yl)methyl or optionally substituted guanidino-C₂₋₄alkyl;
R¹¹ is optionally substituted benzyl;
R¹² is indolyl-3-methyl or optionally substituted benzyl;
R¹³ is optionally substituted benzyl;
R¹⁴ is N-(3-amino-3-oxo-propyl)carboxamide or N-(2-amino-2-oxo-ethyl)carboxamide both optionally substituted;
R^{b}, R^{c}, R^{d}, R^{e}, R^{f}, R^{15a}, R^{15b}, R^{15c}, R^{15d}, R^{15e}, R^{15f}, R^{15g}, R^{15h}, R¹⁵ⁱ, R^{15j}, R^{15k}, R^{15l}, R^{15m}, R¹⁵ⁿ, R^{16a}, R^{16b}, R^{16c} and R^{16d} are in each occurrence independently hydrogen or C₁₋₆alkyl;
R^{a} is hydrogen, C₁₋₆alkyl, hydroxyl or halo;
n and p are each independently 0 to 2; and
q is 0 or 1.

9. The compound of claim 8, wherein:
R¹ is optionally substituted benzyl;
R^{1a} is hydrogen or C₁₋₆alkyl;
R² is (3-methylimidazol-4-yl)methyl;
R³ is C₁₋₆alkyl or hydroxyl-C₁₋₆alkyl;
R^{3a} is hydrogen or C₁₋₆alkyl;
R⁴ is C₁₋₆alkyl;
R⁵ is 3-guanidino-propyl;
R⁶ is 3-guanidino-propyl or amino-C₁₋₆alkyl;
R⁷ is 3-guanidino-propyl or hydroxyl-C₁₋₆alkyl;
R⁸ is optionally substituted benzyl;
R^{8a} is methyl;
R⁹ is optionally substituted benzyl;
R¹⁰ is optionally substituted guanidino-C₂₋₄alkyl, (3-methylimidazol-4-yl)methyl or amino-C₁₋₆alkyl;
R¹¹ is 4-hydroxybenzyl or 4-chlorobenzyl;
R¹² is 1H-indole-3-methyl or 4-chlorobenzyl;
R¹³ is optionally substituted benzyl;
R¹⁴ is —C(═O)NHCH₂C(═O)NH₂;
R^{15a}, R^{15b}, R^{15c}, R^{15d}, R^{15e}, R^{15f}, R^{15g}, R^{15h}, R¹⁵ⁱ, R^{15j}, R^{15k}, R^{15l}, R^{15m}, R¹⁵ⁿ, R^{16a}, R^{16b}, R^{16c} and R^{16d} are hydrogen;
or, a stereoisomer or a pharmaceutically acceptable salt thereof.

10. The compound of either claims 8 or 9, wherein:
R¹ is benzyl;
R^{1a} is hydrogen;
R² is (3-methylimidazol-4-yl)methyl;
R³ is methyl or (1R)-1-hydroxyethyl;
R^{3a} is hydrogen or methyl;
R⁴ is (1S)-1-methylpropyl;
R⁵ is 3-guanidopropyl;
R⁶ is 4-amino-butyl;
R⁷ is 3-guanidopropyl or -(1R)-1-hydroxyethyl;
R⁸ and R^{8a} are methyl;
R⁹ is benzyl;
R¹⁰ is guanidino-propyl, (3-methylimidazol-4-yl)methyl or amino-C₁₋₆alkyl;
R¹¹ is 4-hydroxybenzyl;
R¹² is 1H-indole-3-methyl;
R¹³ is 4-hydroxybenzyl;
R¹⁴ is —C(═O)NHCH₂C(═O)NH₂;
R^{15a}, R^{15b}, R^{15c}, R^{15d}, R^{15e}, R^{15f}, R^{15g}, R^{15h}, R¹⁵ⁱ, R^{15j}, R^{15k}, R^{15l}, R^{15m}, R¹⁵ⁿ, R^{16a}, R^{16b}, R^{16c} and R^{16d} are hydrogen;
or, a stereoisomer or a pharmaceutically acceptable salt thereof.

11. The compound of any of claims 8 to 10, wherein:
R¹ is benzyl;
R^{1a} is hydrogen;
R² is (3-methylimidazol-4-yl)methyl;
R³ is methyl or (1R)-1-hydroxyethyl;
R⁴ is (1S)-1-methylpropyl;
R⁵ is 3-guanidino-propyl;
R⁶ is 4-aminobutyl;
R⁷ is rac-(1R)-1-hydroxyethyl;
R⁸ and R^{8a} are methyl;
R⁹ is benzyl;
R¹⁰ is 3-guanidinopropyl, (3-methylimidazol-4-yl)methyl, aminomethyl, 2-amino-ethyl, 3-aminopropyl or 4 aminobutyl;
R¹¹ is 4-hydroxybenzyl;
R¹² is 1H-indole-3-methyl;
R¹³ is 4-hydroxybenzyl;
R¹⁴ is —C(═O)NHCH₂C(═O)NH.

12. The compound of any of claims 8 to 11, wherein:
R¹ is benzyl;
R^{1a} is hydrogen;
R² is (3-methylimidazol-4-yl)methyl;
R³ is methyl or (1R)-1-hydroxyethyl;
R⁴ is (1S)-1-methylpropyl;
R⁵ is 3-guanidino-propyl;
R⁶ is 4-aminobutyl;
R⁷ is rac-(1R)-1-hydroxyethy;
R⁸ and R^{8a} are methyl;
R⁹ is benzyl;
R¹⁰ is 3-guanidinopropyl:
R¹¹ is 4-hydroxybenzyl;
R¹² is 1H-indole-3-methyl;
R¹³ is 4-hydroxybenzyl;
R¹⁴ is —C(═O)NHCH₂C(═O)NH;
or, a stereoisomer or a pharmaceutically acceptable salt thereof.

13. The compound of any of claims 8 to 11, wherein:
R¹ is benzyl;
R^{1a} is hydrogen;
R² is (3-methylimidazol-4-yl)methyl;
R³ is methyl or (1R)-1-hydroxyethyl;
R⁴ is (1S)-1-methylpropyl;
R⁵ is 3-guanidino-propyl;
R⁶ is 4-aminobutyl;
R⁷ is rac-(1R)-1-hydroxyethy;
R⁵ and R^{8a} are methyl;
R⁹ is benzyl;
R¹⁰ is (3-methylimidazol-4-yl)methyl;
R¹¹ is 4-hydroxybenzyl;
R¹² is 1H-indole-3-methyl;
R¹³ is 4-hydroxybenzyl;
R¹⁴ is —C(═O)NHCH₂C(═O)NH;
or, a stereoisomer or a pharmaceutically acceptable salt thereof.

14. The compound of any of claims 8 to 11, wherein:
R¹ is benzyl;
R^{1a} is hydrogen;
R² is (3-methylimidazol-4-yl)methyl;
R³ is methyl or (1R)-1-hydroxyethyl;
R⁴ is (1S)-1-methylpropyl;
R⁵ is 3-guanidino-propyl;
R⁶ is 4-aminobutyl;
R⁷ is rac-(1R)-1-hydroxyethy;
R⁸ and R^{8a} are methyl;
R⁹ is benzyl;
R¹⁰ is 3-aminopropyl or 4 aminobutyl;
R¹¹ is 4-hydroxybenzyl;
R¹² is 1H-indole-3-methyl;
R¹³ is 4-hydroxybenzyl;
R¹⁴ is —C(═O)NHCH₂C(═O)NH;
or, a stereoisomer or a pharmaceutically acceptable salt thereof.

15. The compound of any of claims 8 to 14 wherein the compound is of formula IIa

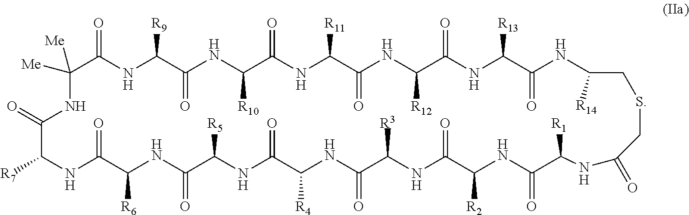

16. The compound according to any one of claims 7 to 15 wherein said compound is selected from TABLE 2.

17. A pharmaceutical composition comprising the compound of any one of claims 1 to 16, or a pharmaceutically acceptable salt, solvate, or stereoisomer thereof, and a pharmaceutically acceptable excipient.

18. Use of a compound of any one of claims 1 to 16, or a pharmaceutically acceptable salt, solvate, or stereoisomer thereof, for preparation of a medicament for the treatment of a bacterial infection in a patient.

19. A method of treatment of a bacterial infection in a mammal, comprising administering to the mammal an effective amount of a compound of any one of claims 1 to 16, or a pharmaceutically acceptable salt, solvate, or stereoisomer thereof, to the mammal at a frequency and for a duration sufficient to provide a beneficial effect to the mammal.

20. A method of treatment of a gram negative mediated infection in a mammal, comprising administering to the mammal an effective amount of a compound of any one of claims 1 to 16, or a pharmaceutically acceptable salt, solvate, or stereoisomer thereof, to the mammal at a frequency and for a duration sufficient to provide a beneficial effect to the mammal.

21. The method of claim 19 or 20, wherein the bacterial infection is an infection involving E. coli.

22. The method of any one of claims 19 to 21 further comprising administering a second therapeutic agent.

23. The method of claim 22, wherein the second therapeutic agent is an aminoglycoside antibiotic, fluoroquinolone antibiotic, β-lactam antibiotic, macrolide antibiotic, glycopeptide antibiotic, rifampicin, chloramphenicol, fluoramphenicol, colistin, mupirocin, bacitracin, daptomycin, or linezolid.
